(19) United States
(12) Patent Application Publication
(10) Pub. No.: US 2024/0024333 A1
(43) Pub. Date: Jan. 25, 2024

(54) USE OF SMALL MOLECULE FAK ACTIVATORS TO PROMOTE MUCOSAL HEALING

(71) Applicants: Regents of the University of Minnesota, Minneapolis, MN (US); University of North Dakota, Grand Forks, ND (US)

(72) Inventors: Marc D. Basson, Grand Forks, ND (US); Vadim J. Gurvich, Apple Valley, MN (US)

(21) Appl. No.: 18/250,493

(22) PCT Filed: Oct. 26, 2021
(86) PCT No.: PCT/US2021/056595

§ 371 Date: Apr. 25, 2023

Related U.S. Application Data
(60) Provisional application No. 63/198,532, filed on Oct. 26, 2020.

(51) Int. Cl. A61K 31/5377; A61P 1/04

(52) U.S. Cl. CPC A61K 31/5377; A61P 1/04

(57) ABSTRACT

Methods to activate or enhance phosphorylation of focal adhesion kinase (FAK) are provided herein. Methods to treat epithelial disorders in mammals, namely diseases of the gut, comprising the administration of one or more small molecules having FAK activation properties are also provided.

### CROSS-REFERENCE TO RELATED APPLICATIONS

This application claims the benefit of priority to U.S. Provisional Appl. Ser. No. 63/198,532, filed Oct. 26, 2020, which is incorporated by reference as if fully set forth herein.

### BACKGROUND

Inadequate intestinal mucosal healing contributes to diverse and common disease states, including peptic ulcer, Crohn's disease, ulcerative colitis, celiac disease, necrotizing enterocolitis, and the loss of the mucosal barrier in critical illness that contributes to bacterial translocation and septic states. The failure to heal a mucosal injury can result in loss of bowel or even life. One example would be the patient hospitalized with an acute flare of Crohn's disease, who is managed medically for a few days with even more aggressive immunosuppression and then taken to surgery if that fails. This not only subjects the patient to a surgical procedure with attendant pain and morbidity, but irretrievably reduces the amount of small intestine available for nutrient absorption. This may ultimately lead to short gut syndrome if subsequent disease flares require repeated resections.

Healing represents an equilibrium between the processes that injure the bowel mucosa (inflammation, ischemia, and luminal agents) and the epithelial sheet migration and proliferation for resurfacing injured gut. However, virtually all current approaches to managing mucosal injury focus on reducing injury (e.g. immunosuppressives, anti-acid agents).

### SUMMARY

The present invention is directed to pharmacological activation of focal adhesion kinase (FAK) using small molecules and the promotion of mucosal healing via the regulation of FAK. For example, provided herein is pharmacological intervention to activate or enhance the phosphorylation of FAK, thereby accelerating mucosal healing. Treatment of epithelial disorders, namely gastrointestinal conditions affecting mucosal surfaces such as Crohn's disease, ulcerative colitis, and peptic ulcer disorder in mammals, is also contemplated herein.

One embodiment provides methods for treating epithelial disorders in a subject in need thereof, namely gut disorders, via the administration of small molecule compounds that promote mucosal healing through the regulation (e.g., positive) of focal adhesion kinase (FAK). Such administration may be accomplished in any number of ways, including without limitation intraperitoneal, intravenous, oral, rectal, or by way of nasogastric or enteric tubes.

Some embodiments provide a method to treat an epithelial disease or a method to activate focal adhesion kinase phosphorylation in eukaryotic cells comprising contacting cells or administering to a subject in need thereof an effective amount of a compound of Formula I or a salt thereof,

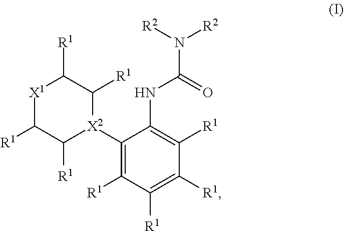

wherein
   X¹ is —C—, —N—, or —O—;
   X² is —N— or —C—; and
   R¹ and R² are independently selected from the group consisting of —H, —OH, and substituted or unsubstituted (C₁-C₂₀)hydrocarbyl, and combinations thereof.

### BRIEF DESCRIPTION OF THE DRAWINGS

The drawings illustrate generally, by way of example, but not by way of limitation, various embodiments discussed in the present document.

FIG. 1 provides a series of graphs and pictures showing in vivo data of 45's effectiveness against ulcers in mice.

FIG. 2 is a graph showing 40's ability to activate FAK.

FIG. 3 is a graph showing 40's Vₘₐₓ for ATP-binding.

FIG. 4 is a graph demonstrating injury scoring in mice being administered different compounds.

FIG. 5 shows the dose-responsive effect of 24 in the in vitro kinase assay, mixing with purified protein of the FAK 35 kD kinase domain.

FIG. 6 is a graph demonstrating that 40 activates FAK within Caco-2 cells as assessed by Western blot for FAK-Y-397 phosphorylation.

FIG. 7 is a graph showing a dose-responsive stimulation of Caco-2 monolayer wound closure in vitro by 40.

FIG. 8 is a graph showing that 10 nM 40 still stimulates Caco-2 monolayer wound closure even in the presence of hydroxyurea to prevent cell proliferation.

FIGS. 9A and 9B, below show the effectiveness of 43 and 42 at various concentrations.

FIGS. 9C and 9D show the effectiveness of 43 and 42 at various concentrations.

FIG. 10 shows the effectiveness of 45 at various concentrations.

### DESCRIPTION

Failure of mucosal healing is central to diseases as diverse as inflammatory bowel disease (IBD), peptic ulcer, and necrotizing enterocolitis (NEC). Failure to heal substantially impairs quality of life in patients with these diseases, may require risky surgery, and even kills patients. Approximately 1 million people in the US are afflicted with IBD (1), and 51,200 died of IBD in the US alone in 2013 (2). Healing of GI mucosal injury represents an equilibrium between injurious agents and the migration and proliferation of epithelial cells at the wound edge. Most treatments, such as antacids or antisecretory drugs for ulcers, anti-inflammatory agents for IBD, attempt to minimize further injury. However, intact mucosa resists noxious stimuli more effectively than mucosa in which the mucosal barrier has been breached. Therefore, promoting mucosal healing by epithelial sheet migration is a potentially synergistic target. Surprisingly, despite significant research carried out in the area of factors and cytokines, there are no therapeutic agents promoting mucosal healing directly in treating IBD, peptic ulcer, NEC, or other gut mucosal lesions. Therefore, developing a new therapy to promote mucosal healing is an important unmet medical need affecting a substantial number of patients both nationally and worldwide.

Healing of GI mucosal injury represents an equilibrium between injurious agents and the migration and proliferation of epithelial cells at the wound edge. Most treatment attempts to minimize further injury (e.g. antacids or anti-secretory drugs for ulcers, anti-inflammatory agents for IBD). However, intact mucosa resists noxious stimuli more effectively than mucosa in which the mucosal barrier has been breached, so promoting mucosal healing by epithelial sheet migration is a potentially synergistic target. Surprisingly, despite all of the scientific work that has been done with growth factors and cytokines, no currently available therapeutic agents promote mucosal healing directly in treating IBD, peptic ulcer, NEC, or other gut mucosal lesions.

Focal adhesion kinase (FAK), a nonreceptor protein tyrosine kinase, is expressed in most tissues and cell types and is highly conserved across mammalian and other eukaryotic species (Schaller MD (2010) J Cell Sci 123:1007-1013). The phosphorylation of FAK's tyrosine and serine residues in response to integrin engagement, mitogenic neuropeptides, lysophosphatidic acid, platelet-derived growth factor, activated Rho, and selected oncogenes leads to the formation of docking sites for a variety of signaling molecules that may regulate cell morphology, locomotion, proliferation, differentiation, and apoptosis (Schaller MD; Parsons JT (2003) J Cell Sci 116:1409-1416; Hanks SK, Polte TR (1997) Bioessays 19:137-145).

Focal adhesion kinase (FAK) is a regulator of epithelial sheet migration. FAK activation is a convergent target for many growth factors (3) and inhibiting (4) or reducing (5) FAK inhibits migration. However, activated FAK is decreased in migrating intestinal epithelial cells in vitro (4) and at the edge of human mucosal ulcers (6), making FAK an attractive target to promote mucosal healing. While searching for small molecules that would mimic a subdomain of the N-terminal FERM domain of FAK and therefore competitively inhibit FAK-AKT binding (7), two small molecules were identified that actually activate FAK at concentrations as low as 10 nM. Follow-up structure-activity relationship study using commercially available analogs (SAR by commerce) identified additional compounds that similarly activate FAK at nanomolar to picomolar levels, as well as a structural framework for compounds with such activity. The compounds investigated for further testing promoted epithelial sheet migration in vitro. In vivo testing was also carried out. The compounds markedly accelerated mucosal ulcer healing in two mouse models without obvious toxicity. It is believed that these compounds mimic part of the FERM domain and act by interfering with the FAK FERM domain inhibition of the FAK kinase domain. These compounds provide for a novel treatment to promote mucosal healing in diseases such as IBD, peptic ulcer, ischemic colitis, and NEC.

Mucosal healing involves epithelial motility, proliferation, and differentiation. Focal adhesion kinase (FAK) influences all three. FAX is an autophosphorylating tyrosine kinase that mediates downstream signals by receptors for matrix proteins and many growth factors and can promote epithelial cell motility, a first step in mucosal healing. Many studies have focused on FAK activation within minutes after ligand binding to FAK-associated membrane receptors. Studies have suggested that FAK may be regulated at the protein level as well as in its phosphorylation during gut epithelial cell motility in vitro and during mucosal healing in vivo.

Little has heretofore been known about the regulation of FAK protein levels. It has been hypothesized that integrin- and FAK-related signal events regulate FAK protein pools at the mRNA level, by modulating FAK gene transcription or FAK mRNA degradation, and that TGFβ stimulates FAK by acting on this pathway. A control point for regulation of intracellular FAX protein and mRNA pools during intestinal epithelial motility has been characterized, and the role of protein levels of this molecule in gut epithelial wound healing has been demonstrated. A previously unknown pathway by which intestinal epithelial cell motility is regulated and its specific modulation by TGFβ has also been characterized.

Provided herein are FAK-activating agents/compounds that can specifically promote epithelial restitution and mucosal healing, thereby treating/healing mucosal injury either without or in synergistic combination with immunosuppressives.

### Compounds

Provided herein is the further development of the observation that certain small molecules/compounds that mimic the tertiary structure of one subdomain of FAK result in its increased activation (5). No currently available therapeutic specifically activates FAK, although numerous growth factors and cytokines are noted to activate FAK along with many other signals within the cell. No currently available agent directly and specifically promotes intestinal epithelial sheet migration and mucosal healing. While tyrosine phosphatase inhibitors are known, and indeed Novartis has a SHP-2 inhibitor in clinical trials for cancer (8), the molecules/compounds provided herein do not appear to exert their actions by inhibiting tyrosine phosphatases (low levels of inhibition may occur) which would be expected to result in the activation of multiple kinases other than FAK but appear to promote the activation of FAK itself. (Indeed, preliminary data shows SHP-2 is not inhibited and a PTP-PEST inhibitor does not block the effect.) This selectivity permits substantially higher doses with less toxicity.

Also provided herein are compounds of the following formula(s):

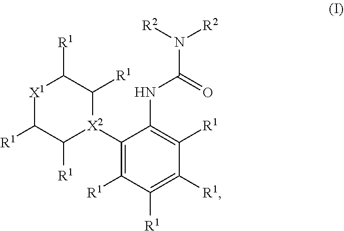

wherein
   X¹ is —C—, —N—, or —O—;
   X² is —N— or —C—; and
   R¹ and R² are independently selected from the group consisting of —H, —OH, and substituted or unsubstituted (C₁-C₂₀)hydrocarbyl, and combinations thereof.

In some embodiments, the (C₁-C₂₀)hydrocarbyl is chosen from (C₁-C₂₀)alkyl, (C₁-C₂₀)alkenyl, (C₁-C₂₀)alkynyl, (C₁-C₂₀)acyl, (C₁-C₂₀)cycloalkyl, (C₁-C₂₀)aryl, (C₁-C₂₀)alkoxy, (C₁-C₂₀)haloalkyl, and combinations thereof.

In some embodiments, the compound is represented by Formula II:

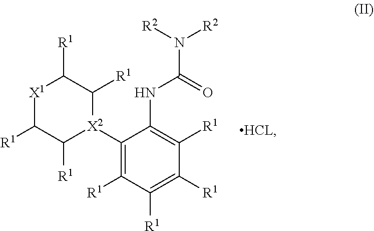

wherein
   X¹ is —C—, —N—, or —O—;
   X² is —N— or —C—; and
   R¹ and R² are independently selected from the group consisting of —H, —F, —OH, substituted or unsubstituted (C₁-C₂₀) hydrocarbyl, and combinations thereof. The (C₁-C₂₀)hydrocarbyl is selected from the group consisting of (C₁-C₂₀)alkyl, (C₁-C₂₀)alkenyl, (C₁-C₂₀)alkynyl, (C₁-C₂₀)acyl, (C₁-C₂₀)cycloalkyl, (C₁-C₂₀)aryl, (C₁-C₂₀)alkoxy, (C₁-C₂₀)haloalkyl, and combinations thereof.

In some embodiments, the compound is represented by Formula III:

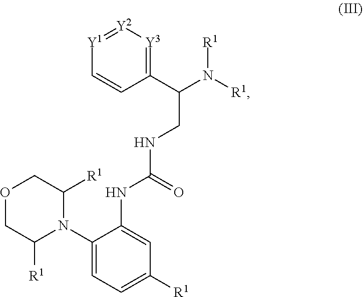

wherein
   at each occurrence, R¹ is independently selected from the group consisting of —H, —F, —CF₃, —CH₃, —CH₂CH₃, —OCH₃, —OCH₂CH₃, and combinations thereof; and
      Y¹, Y², and Y³ are independently selected from the group consisting of —C—, —N—, —O—, and combinations thereof.

In some embodiments, the compound is represented by Formula IV:

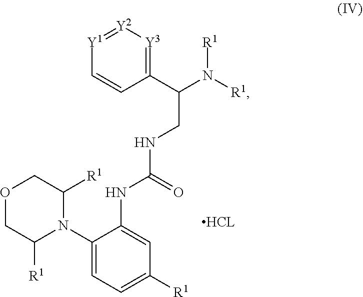

Wherein at each occurrence, R¹ is independently selected from the group consisting of H, —F, —CF₃, —CH₃, —CH₂CH₃, —OCH₃, —OCH₂CH₃, and combinations thereof; and
Y¹, Y², and Y³ are independently selected from the group consisting of —N—, —C—, —O—, and combinations thereof.

In some embodiments, the compound is represented by Formula V:

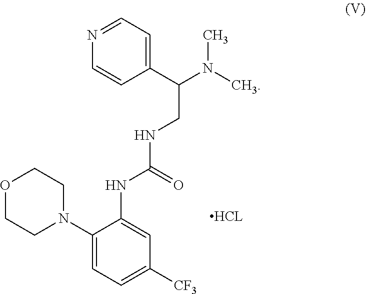 [SMILES: CN(C)C(CNC(=O)Nc1cc(C(F)(F)F)ccc1N1CCOCC1)c1ccncc1]

In some embodiments, the compound is represented by Formula VI:

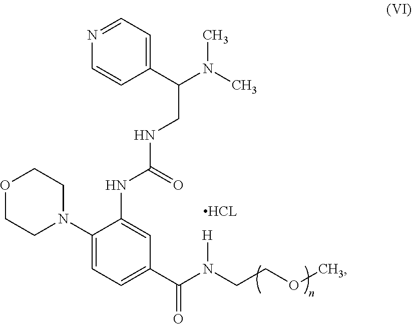

wherein n is any positive integer.

In some embodiments, the compound is represented by Formula VII:

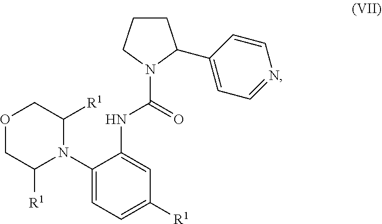

wherein at each occurrence, R¹ is independently selected from the group consisting of —H, —F, —CF₃, —CH₃, —CH₂CH₃, —OCH₃, —OCH₂CH₃, and combinations thereof.

In some embodiments, the compound is represented by Formula VIII:

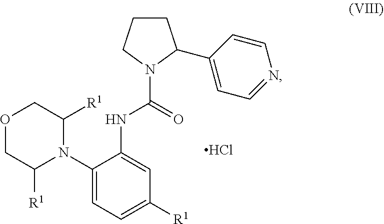

wherein at each occurrence, R¹ is independently selected from the group consisting of —H, —F, —CF₃, —CH₃, —CH₂CH₃, —OCH₃, —OCH₂CH₃, and combinations thereof.

Some embodiments provide for combination therapy, such as FAK activation small molecules and immunotherapy (e.g., immunosuppressives; immunotherapy) and/or anti-ulcer therapy (e.g., anti-acid secretory agents). For example, oral administration, either by direct dosing or via an enteral release formulation, could be synergistic with conventional immunosuppressive therapy in IBD, enhancing quality of life at lower immunosuppressive dosing.

In the paradigmatic case of the patient whose Crohn's disease has flared, adding a parenteral drug that activates enterocytic FAK for three days during aggressive immunosuppression can make the difference between hospital discharge and surgery.

### Pharmaceutical Compositions/Administration/Disorders

The present disclosure also contemplates pharmaceutical compositions comprising one or more compounds disclosed herein, one or more pharmaceutically acceptable carriers, diluents, excipients or combinations thereof. A “pharmaceutical composition” refers to a chemical or biological composition suitable for administration to a subject (e.g., mammal). Such compositions can be specifically formulated for administration via one or more of a number of routes, including but not limited to buccal, cutaneous, epicutaneous, epidural, infusion, inhalation, intraarterial, intracardial, intracerebroventricular, intradermal, intramuscular, intranasal, intraocular, intraperitoneal, intraspinal, intrathecal, intravenous, oral, parenteral, pulmonary, rectally via an enema or suppository, subcutaneous, subdermal, sublingual, transdermal, and transmucosal. In addition, administration can by means of capsule, drops, foams, gel, gum, injection, liquid, patch, pill, porous pouch, powder, tablet, or other suitable means of administration.

A “pharmaceutical excipient” or a “pharmaceutically acceptable excipient” comprises a carrier, sometimes a liquid, in which an active therapeutic agent is formulated. The excipient generally does not provide any pharmacological activity to the formulation, though it may provide chemical and/or biological stability, and release characteristics. Examples of suitable formulations can be found, for example, in Remington, The Science and Practice of Pharmacy, 20th Edition, (Gennaro, A. R., Chief Editor), Philadelphia College of Pharmacy and Science, 2000, which is incorporated by reference in its entirety.

As used herein “pharmaceutically acceptable carrier” or “excipient” includes any and all solvents, dispersion media, coatings, antibacterial and antifungal agents, isotonic and absorption delaying agents that are physiologically compatible. The carrier is suitable for, among other applications, parenteral administration. Alternatively, the carrier can be suitable for intravenous, intraperitoneal, intramuscular, sublingual, or oral administration. Pharmaceutically acceptable 1.5 carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the active compound, use thereof in the pharmaceutical compositions is contemplated. Supplementary active compounds can also be incorporated into the compositions.

Pharmaceutical compositions can be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (e.g., glycerol, propylene glycol, and liquid polyethylene glycol), and suitable mixtures thereof. The proper fluidity can be maintained, for example, by the use of a coating such as lecithin, by the maintenance of the required particle size in the case of dispersion and by the use of surfactants.

In many cases, it will be preferable to include isotonic agents, for example, sugars, polyalcohols such as mannitol, sorbitol, or sodium chloride in the composition. Prolonged absorption of the injectable compositions can be brought about by including in the composition an agent which delays absorption, for example, monostearate salts and gelatin. Moreover, the compounds described herein can be formulated in a time release formulation, for example in a composition that includes a slow release polymer. The active compounds can be prepared with carriers that will protect the compound against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid and polylactic, polyglycolic copolymers (PLG). Many methods for the preparation of such formulations are known to those skilled in the art.

Oral forms of administration are also contemplated herein. The pharmaceutical compositions can be orally administered as a capsule (hard or soft), tablet (film coated, enteric coated or uncoated), powder or granules (coated or uncoated) or liquid (solution or suspension). The formulations can be conveniently prepared by any of the methods well-known in the art. The pharmaceutical compositions can include one or more suitable production aids or excipients including fillers, binders, disintegrants, lubricants, diluents, flow agents, buffering agents, moistening agents, preservatives, colorants, sweeteners, flavors, and pharmaceutically compatible carriers.

The compounds can be administered by a variety of dosage forms as known in the art. Any biologically-acceptable dosage form known to persons of ordinary skill in the art, and combinations thereof, are contemplated. Examples of such dosage forms include, without limitation, chewable tablets, quick dissolve tablets, effervescent tablets, reconstitutable powders, elixirs, liquids, solutions, suspensions, emulsions, tablets, multi-layer tablets, bi-layer tablets, capsules, soft gelatin capsules, hard gelatin capsules, caplets, lozenges, chewable lozenges, beads, powders, gum, granules, particles, microparticles, dispersible granules, cachets, douches, suppositories, creams, topicals, inhalants, aerosol inhalants, patches, particle inhalants, implants, depot implants, ingestibles, injectables (including subcutaneous, intramuscular, intravenous, and intradermal), infusions, and combinations thereof.

Other compounds which can be included by admixture are, for example, medically inert ingredients (e.g., solid and liquid diluent), such as lactose, dextrosesaccharose, cellulose, starch or calcium phosphate for tablets or capsules, olive oil or ethyl oleate for soft capsules and water or vegetable oil for suspensions or emulsions; lubricating agents such as silica, talc, stearic acid, magnesium or calcium stearate and/or polyethylene glycols; gelling agents such as colloidal clays; thickening agents such as gum tragacanth or sodium alginate, binding agents such as starches, arabic gums, gelatin, methylcellulose, carboxymethylcellulose or polyvinylpyrrolidone; disintegrating agents such as starch, alginic acid, alginates or sodium starch glycolate; effervescing mixtures; dyestuff; sweeteners; wetting agents such as lecithin, polysorbates or laurylsulphates; and other therapeutically acceptable accessory ingredients, such as humectants, preservatives, buffers and antioxidants, which are known additives for such formulations.

Liquid dispersions for oral administration can be syrups, emulsions, solutions, or suspensions. The syrups can contain as a carrier, for example, saccharose or saccharose with glycerol and/or mannitol and/or sorbitol. The suspensions and the emulsions can contain a carrier, for example a natural gum, agar, sodium alginate, pectin, methylcellulose, carboxymethylcellulose, or polyvinyl alcohol.

The amount of active compound in a therapeutic composition can vary according to factors such as the disease state, age, gender, weight, patient history, risk factors, predisposition to disease, administration route, pre-existing treatment regime (e.g., possible interactions with other medications), and weight of the individual. Dosage regimens can be adjusted to provide the optimum therapeutic response. For example, a single bolus can be administered, several divided doses can be administered over time, or the dose can be proportionally reduced or increased as indicated by the exigencies of therapeutic situation.

“Dosage unit form,” as used herein, refers to physically discrete units suited as unitary dosages for the mammalian subjects to be treated, each unit containing a predetermined quantity of active compound calculated to produce the desired therapeutic effect in association with the required pharmaceutical carrier. The specification for the dosage unit forms are dictated by and 1.5 can be directly dependent on the unique characteristics of the active compound and the particular therapeutic effect to be achieved, and the limitations inherent in the art of compounding such an active compound for the treatment of sensitivity in individuals. In therapeutic use for treatment of conditions in mammals (e.g., humans) for which the compounds disclosed herein, or an appropriate pharmaceutical composition thereof are effective, the compounds disclosed herein can be administered in an effective amount. The dosages as suitable for this disclosure can be a composition, a pharmaceutical composition or any other compositions described herein.

The dosage can be administered once, twice, thrice or four times a day, although more frequent dosing intervals are possible. The dosage can be administered every day, every 2 days, every 3 days, every 4 days, every 5 days, every 6 days, and/or every 7 days (once a week). The dosage can be administered daily for up to and including 30 days, preferably between 7-10 days. Or the dosage can be administered twice a day for 10 days. If the patient requires treatment for a chronic disease or condition, the dosage can be administered for as long as signs and/or symptoms persist. The patient may require “maintenance treatment” where the patient is receiving dosages every day for months, years, or the remainder of their lives. In addition, the composition can affect prophylaxis of recurring symptoms. For example, the dosage can be administered once or twice a day to prevent the onset of symptoms in patients at risk, especially for asymptomatic patients.

The compositions described herein can be administered in any of the following routes: buccal, epicutaneous, epidural, infusion, inhalation, intraarterial, intracardial, intracerebroventricular, intradermal, intramuscular, intranasal, intraocular, intraperitoneal, intraspinal, intrathecal, intravenous, oral, parenteral, pulmonary, rectally via an enema or suppository, subcutaneous, subdermal, sublingual, transdermal, and transmucosal. The administration can be local, where the composition is administered directly, close to, in the locality, near, at, about, or in the vicinity of, the site(s) of disease, e.g., inflammation, or systemic, wherein the composition is given to the patient and passes through the body widely, thereby reaching the site(s) of disease. Local administration can be administration to the cell, tissue, organ, and/or organ system, which encompasses and/or is affected by the disease, and/or where the disease signs and/or symptoms are active or are likely to occur. Administration can be topical with a local effect, composition is applied directly where its action is desired. Administration can be enteral wherein the desired effect is systemic (non-local), composition is given via the digestive tract. Administration can be parenteral, where the desired effect is systemic, composition is given by other routes than the digestive tract.

Also contemplated herein are compositions comprising a therapeutically effective amount of one or more compounds provided herein that are useful in a method for treating an epithelial 1.5 disease, such as a gut disorder, including but not limited to Crohn's disease, celiac disease, peptic ulcer disease, IBD and/or ulcerative colitis, necrotizing enterocolitis (NEC), or loss of mucosal barrier, for example in illness that contributes to bacterial translocation and septic states, or to promote skin wound epithelialization, oral ulcer healing, or other epithelial wound healing disorders of skin or cornea (e.g., eczema, psoriasis, epithelial carcinoma, asthma and/or corneal abrasions).

The term “therapeutically effective amount” as used herein, refers to that amount of one or more compounds disclosed herein that elicits a biological or medicinal response in a tissue system, animal or human, that is being sought by a researcher, veterinarian, medical doctor or other clinician, which includes alleviation of the symptoms of the disease or disorder being treated. The therapeutically effective amount can be that which may treat or alleviate the disease or symptoms of the disease at a reasonable benefit/risk ratio applicable to any medical treatment. However, it is to be understood that the total daily usage of the compounds and compositions described herein can be decided by the attending physician within the scope of sound medical judgment. The specific therapeutically-effective dose level for any particular patient will depend upon a variety of factors, including the condition being treated and the severity of the condition; activity of the specific compound employed; the specific composition employed; the age, body weight, general health, gender and diet of the patient: the time of administration, route of administration, and rate of excretion of the specific compound employed; the duration of the treatment; drugs used in combination or coincidentally with the specific compound employed; and like factors well known to the researcher, veterinarian, medical doctor or other clinician. It is also appreciated that the therapeutically effective amount can be selected with reference to any toxicity, or other undesirable side effect, that might occur during administration of one or more of the compounds described herein.

Values expressed in a range format should be interpreted in a flexible manner to include not only the numerical values explicitly recited as the limits of the range, but also to include all the individual numerical values or sub-ranges encompassed within that range as if each numerical value and sub-range were explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. In addition, it is to be understood that the phraseology or terminology employed herein, and not otherwise defined, is for the purpose of description only and not of limitation. Any use of section headings is intended to aid reading of the document and is not to be interpreted as limiting. Further, information that is relevant to a section heading may occur within or outside of that particular section. Furthermore, all publications, patents, and patent documents referred to in this document are incorporated by reference herein in their entirety, as though individually incorporated by reference.

In the methods described herein, the steps can be carried out in any order without departing from the spirit of this disclosure, except when a temporal or operational sequence is explicitly recited. Furthermore, specified steps can be carried out concurrently unless explicit claim language recites that they be carried out separately. For example, a claimed step of doing X and a claimed step of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range.

The term “organic group” as used herein refers to any carbon-containing functional group. Examples can include an oxygen-containing group such as an alkoxy group, aralkyloxy group, a carboxyl group including a carboxylic acid, carboxylate, and a carboxylate ester; a sulfur-containing group such as an alkyl and aryl sulfide group; and other heteroatom-containing groups. Non-limiting examples of organic groups include OR, OOR, OC(O)N(R)₂, CN, CF₃, OCF₃, R, C(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, C(═NOR)R, and substituted or unsubstituted (C₁-C₁₀₀)hydrocarbyl, wherein R can be hydrogen (in examples that include other carbon atoms) or a carbon-based moiety, and wherein the carbon-based moiety can be substituted or unsubstituted.

The term “substituted” as used herein in conjunction with a molecule or an organic group as defined herein refers to the state in which one or more hydrogen atoms contained therein are replaced by one or more non-hydrogen atoms. The term “functional group” or “substituent” as used herein refers to a group that can be or is substituted onto a molecule or onto an organic group. Examples of substituents or functional groups include, but are not limited to, a halogen (e.g., F, Cl, Br, and I); an oxygen atom in groups such as hydroxy groups, alkoxy groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxyamines, nitrites, nitro groups, N-oxides, hydrazides, azides, and enamines; and other heteroatoms in various other groups. Non-limiting examples of substituents that can be bonded to a substituted carbon (or other) atom include F, Cl, Br, I, OR, OC(O)N(R)₂, CN, NO, NO₂, ONO₂, azido, CF₃, OCF₃, R, O(oxo), S (thiono), C(O), S(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, and C(═NOR)R, wherein R can be hydrogen or a carbon-based moiety; for example, R can be hydrogen, (C₁-C₁₀₀)hydrocarbyl, alkyl, acyl, cycloalkyl, aryl; or wherein two R groups bonded to a nitrogen atom or to adjacent nitrogen atoms can together with the nitrogen atom or atoms form a heterocyclyl.

The term “alkyl” as used herein refers to straight chain and branched alkyl groups and cycloalkyl groups having from 1 to 40 carbon atoms, 1 to about 20 carbon atoms, 1 to 12 carbons or, in some embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “alkenyl” as used herein refers to straight and branched chain and cyclic alkyl groups as defined herein, except that at least one double bond exists between two carbon atoms. Thus, alkenyl groups have from 2 to 40 carbon atoms, or 2 to about 20 carbon atoms, or 2 to 12 carbon atoms or, in some embodiments, from 2 to 8 carbon atoms. Examples include, but are not limited to vinyl, —CH═CH(CH₃), —CH═C(CH₃)₂, —C(CH₃)═CH₂, —C(CH₃)═CH(CH₃), —C(CH₂CH₃KH₂, cyclohexenyl, cyclopentenyl, cyclohexadienyl, butadienyl, pentadienyl, and hexadienyl among others.

The term “alkynyl” as used herein refers to straight and branched chain alkyl groups, except that at least one triple bond exists between two carbon atoms. Thus, alkynyl groups have 1.5 from 2 to 40 carbon atoms, 2 to about 20 carbon atoms, or from 2 to 12 carbons or, in some embodiments, from 2 to 8 carbon atoms. Examples include, but are not limited to —CH, —C≡C(CH₃), —C≡C(CH₂CH₃), —CH₂C≡CH, —CH₂C≡C(CH₃), and —CH₂C≡C(CH₂CH₃) among others.

The term “acyl” as used herein refers to a group containing a carbonyl moiety wherein the group is bonded via the carbonyl carbon atom. The carbonyl carbon atom is bonded to a hydrogen forming a “formyl” group or is bonded to another carbon atom, which can be part of an alkyl, aryl, aralkyl cycloalkyl, or cycloalkylalkyl. An acyl group can include 0 to about 12, 0 to about 20, or 0 to about 40 additional carbon atoms bonded to the carbonyl group. An acyl group can include double or triple bonds within the meaning herein. An acryloyl group is an example of an acyl group. An acyl group can also include heteroatoms within the meaning herein. A nicotinoyl group (pyridyl-3-carbonyl) is an example of an acyl group within the meaning herein. Other examples include acetyl, benzoyl, phenylacetyl, pyridylacetyl, cinnamoyl, and acryloyl groups and the like. When the group containing the carbon atom that is bonded to the carbonyl carbon atom contains a halogen, the group is termed a “haloacyl” group. An example is a trifluoroacetyl group.

The term “cycloalkyl” as used herein refers to cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined herein. Representative substituted cycloalkyl groups can be mono-substituted or substituted more than once, such as, but not limited to, 2,2-, 2,3-, 2,4-2,5- or 2,6-disubstituted cyclohexyl groups or mono-, di- or tri-substituted norbornyl or cycloheptyl groups, which can be substituted with, for example, amino, hydroxy, cyano, carboxy, nitro, thio, alkoxy, and halogen groups. The term “cycloalkenyl” alone or in combination denotes a cyclic alkenyl group.

The term “aryl” as used herein refers to cyclic aromatic hydrocarbon groups that do not contain heteroatoms in the ring. Thus, aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons in the ring portions of the groups. Aryl groups can be unsubstituted or substituted, as defined herein. Representative substituted aryl groups can be mono-substituted or substituted more than once, such as, but not limited to, a phenyl group substituted at any one or more of 2-, 3-, 4-, 5-, or 6-positions of the phenyl ring, or a naphthyl group substituted at any one or more of 2- to 8-positions thereof.

The term “alkoxy” as used herein refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can include about 1 to about 12, about 1 to about 20, or about 1 to about 40 carbon atoms bonded to the oxygen atom, and can further include double or triple bonds, and can also include heteroatoms. For example, an allyloxy group or a methoxyethoxy group is also an alkoxy group within the meaning herein, as is a methylenedioxy group in a context where two adjacent atoms of a structure are substituted therewith.

The term “amine” as used herein refers to primary, secondary, and tertiary amines having, e.g., the formula N(group)₃ wherein each group can independently be H or non-H, such as alkyl, aryl, and the like. Amines include but are not limited to R—NH₂, for example, alkylamines, arylamines, alkylarylamines; R₂NH wherein each R is independently selected, such as dialkylamines, diarylamines, aralkylamines, and the like; and R₃N wherein each R is independently selected, such as trialkylamines, di alkylarylamines, alkyldiarylamines, triarylamines, and the like. The term “amine” also includes ammonium ions as used herein.

The term “haloalkyl” group, as used herein, includes mono-halo alkyl groups, poly-halo alkyl groups wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, 1,2-dichloroethyl, 1,3-dibromo-3,3-difluoropropyl, perfluorobutyl, and the like.

As used herein, the term “hydrocarbyl” refers to a functional group derived from a straight chain, branched, or cyclic hydrocarbon, and can be alkyl, alkenyl, alkynyl, aryl, cycloalkyl, acyl, or any combination thereof. Hydrocarbyl groups can be shown as (Cₐ-C_{b})hydrocarbyl, wherein a and b are integers and mean having any of a to b number of carbon atoms. For example, (C₁-C₄)hydrocarbyl means the hydrocarbyl group can be methyl (C₁), ethyl (C₂), propyl (C₃), or butyl (C₄), and (C₀-C_{b})hydrocarbyl means in certain embodiments there is no hydrocarbyl group.

As used herein, the term “salts” and “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic groups such as amines; and alkali or organic salts of acidic groups such as carboxylic acids. Pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, and nitric; and the salts prepared from organic acids such as acetic, propionic, succinic, glycolic, stearic, lactic, malic, tartaric, citric, ascorbic, pamoic, malefic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic, and the like.

Pharmaceutically acceptable salts can be synthesized from the parent compound which contains a basic or acidic moiety by conventional chemical methods. In some instances, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, the disclosure of which is hereby incorporated by reference.

The term “solvate” means a compound, or a salt thereof, that further includes a stoichiometric or non-stoichiometric amount of solvent bound by non-covalent intermolecular forces. Where the solvent is water, the solvate is a hydrate.

Although parenteral dosing would be appropriate for acute treatment, oral administration would clearly be of value in the chronic setting when such molecules are applied to chronic disorders of mucosal healing such as BD or celiac disease in the outpatient setting. Luminal administration of these agents would be clinically advantageous in outpatients, either orally or rectally for proctitis, as is now done with Proctofoam enemas.

### Screening Additional Compounds

Potential drug candidates can be screened/tested in a biochemical assay to assess the potency to stimulate Caco-2 (by Western blotting for FAK-397) and HIEC-6 FAK activation (by a commercially available ELISA for FAK-Y-397). To validate their activity, the assay will be performed by two different techniques in two different cell lines. Successful molecules will be further confirmed that Caco-2 monolayer wound closure is enhanced in parallel studies since HIEC-6 cells don't form tight monolayers or display epithelial sheet migration. This longer experiment (24 hours vs. 1 hour) will also offer an initial screen for unexpected potent 1.5 cytotoxicity. The most successful molecules will be submitted for ADMET assays (including metabolic stability, solubility (pH 7.4), Caco-2 (permeability) and log D determination).

Further assays to screen compounds can include the following:

The specificity of candidate molecules for the activation of FAK vs. other tyrosine kinases can be determined (A). Further, whether they modulate relevant tyrosine phosphatases can be assessed (B).

A. Tyrosine Kinase Studies

i. Members of the Jak family of tyrosine kinases, kinases such as Src, Pyk2, Jak1 and/or Jak2 are tyrosine kinases of the class that like FAK are not membrane-bound. Caco-2 cells will be treated with potential candidate compounds (10 nM) or relevant vehicle (DMSO) control. Activation of FAK, Src, and Jak1 can be assessed by Western blotting with phosphospecific antibodies to FAK-Y-397, Src-Y-419, and Jak1-Y1034/1035 that indicate the activation of these proteins (25, 26).

ii. If Src or Jak1 phosphorylation appears increased by experiment Ai above, then true activation by direct action, indirect activation consequent to FAK activation, or some other phosphorylation event will be distinguished/determined. To determine this in vitro kinase assays can be performed using commercially available purified human FAK for the compound(s) at 0, 1, 10, 30, 70, 100 pM and compare the effects to the effects of similar concentrations on Src and Jak1 activity using a purified synthetic substrate.

iii. If Src or Jak1 phosphorylation is increased in Ai, but in vitro kinase activity is not changed in Aii, then the possibility that the effect in intact cells represents a consequence of FAK activation by these molecules will be considered and will be tested, for example, by simultaneously using siRNA to knock down FAK during treatment with the compounds (e.g., FAK activators) and then investigate Src or Jak1 phosphorylation as appropriate.

B. Tyrosine Phosphatase Studies

a. 45 does not activate SHP2. Via a similar and published (24) assay kit (Anaspec #AS-7100), the effects of 45 and/or other compounds/candidate FAK activators will be tested on PTP1B and PTP-PEST tyrosine phosphatase activity at concentrations 0.1×, 1×, 10×, and 100× the lowest concentration required to activate FAK within intact Caco-2 cells.

b. FAK will be immuno-precipitated and Western blot for SHP2, PTP-PEST, and PTP1B after treatment of Caco-2 cells with 45 and/or other compounds/candidate FAK activators or a DMSO vehicle control.

### Examples

The following examples are offered by way of illustration. But the present disclosure is not limited to the examples given herein.

### Introduction

IBD (including Crohn's disease and ulcerative colitis) is a chronic GI idiopathic inflammatory disorder afflicting over one million Americans. Inadequate mucosal healing causes many common diseases in addition to IBD. Those include peptic ulcer, celiac disease, NEC, and the mucosal barrier failure in critical illness that leads to sepsis. Failure to heal a mucosal injury can result in loss of bowel or even life. One paradigmatic example would be the patient hospitalized with an acute flare of Crohn's disease, who is managed medically for a few days with even more aggressive immunosuppression and then taken to surgery if that fails. This not only subjects the patient to risky surgery with attendant pain and morbidity, but irretrievably reduces the amount of small intestine available for nutrient absorption. This may ultimately lead to short gut syndrome if subsequent disease flares require repeated resections.

Healing represents an equilibrium between the processes that injure the bowel mucosa (inflammation, ischemia, and lumina) agents) and the epithelial sheet migration and proliferation required to resurface injured gut. However, virtually all current approaches to managing mucosal injury focus on reducing injury (e.g., immunosuppressives, anti-acid agents). The only therapeutic that may promote healing is sucralfate, which some have hypothesized to bind luminal growth factors and plaster them across peptic ulcers.

FAK activation plays a role in mucosal healing. Therefore, developing a new therapy for GI mucosal healing is an important unmet medical need affecting millions of patients nationally and worldwide. Provided herein are FAK-activating agents compounds that can specifically promote epithelial restitution and mucosal healing, offering the ability to heal mucosal injury either without or in synergistic combination with immunosuppressives. In the paradigmatic case of the patient whose Crohn's disease has flared, adding a parenteral drug that activates enterocytic FAK for three days during aggressive immunosuppression can make the difference between hospital discharge and surgery.

This novel approach builds upon the observation that certain small molecules that mimic the tertiary structure of one subdomain of FAK result in FAK activation. No known therapeutic agent specifically activates FAK, although diverse growth factors and cytokines activate FAK along with many other kinases. No currently known agent directly and specifically promotes intestinal epithelial sheet migration and mucosal healing except, as noted above, for the idea that sucralfate promotes peptic ulcer healing not only by “bandaging” ulcers, but by creating a poultice with luminal growth factors. Tyrosine phosphatase inhibitors are known. For instance, a SHP-2 inhibitor is in clinical trials for cancer. However, the molecules provided herein are not tyrosine phosphatase inhibitors (which would activate many kinases other than FAK) but promote the activation of FAK itself. (Preliminary data shows neither Src nor Pyk2 is activated, neither SHP-2 activity nor PTP-1B-FAK association are inhibited, and a PTP-PEST inhibitor does not block the effect.) Such selectivity permits substantially higher doses with less toxicity.

### Example 1

While searching for small molecules that would mimic a key subdomain of the N-terminal FERM domain of FAK and therefore competitively inhibit FAK-AKT binding, 2 small molecules (9 and 10) were identified that actually activate FAK at concentrations as low as 10 nM. Biological evaluation of 9, in vitro, suggested that it potently activates FAK and promotes intestinal epithelial sheet migration in monolayer wound closure. For example, 9 specifically increased FAK-Tyr 397 phosphorylation within intact cells, while activating neither Pyk2, a close paralog of FAK, nor Src, another key non-receptor kinase within the focal adhesion complex, respectively. 9 stimulated monolayer wound closure in vitro, and intestinal mucosal healing in vivo. The in vitro effects on wound closure were blocked by FAK inhibition, while the in vivo effects were accompanied by increased FAK activation at the ulcer edge. Encouraged by this FAK activation by 9, we designed and implemented follow-up structure-activity relationship (SAR) studies using commercially available analogs (1-13(SAR by commerce). These compounds show high structural similarity to 9 and suitable in silico predicted drug-like properties.

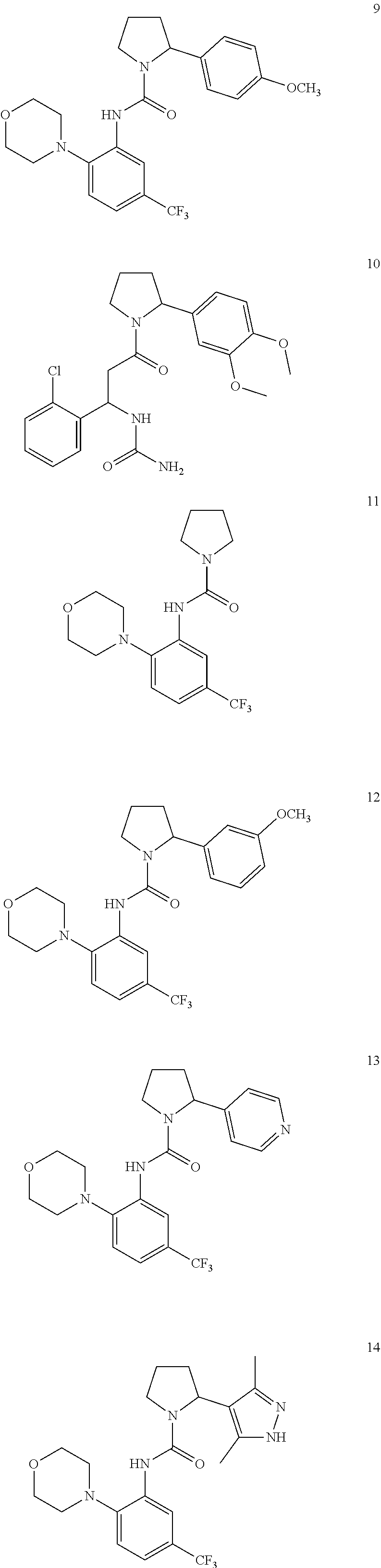 [SMILES: COc1ccc(C2CCCN2C(=O)Nc2cc(C(F)(F)F)ccc2N2CCOCC2)cc1 ; COc1ccc(C2CCCN2C(=O)CC(NC(N)=O)c2ccccc2Cl)cc1OC ; O=C(Nc1cc(C(F)(F)F)ccc1N1CCOCC1)N1CCCC1 ; COc1cccc(C2CCCN2C(=O)Nc2cc(C(F)(F)F)ccc2N2CCOCC2)c1 ; O=C(Nc1cc(C(F)(F)F)ccc1N1CCOCC1)N1CCCC1c1ccncc1 ; Cc1n[nH]c(C)c1C1CCCN1C(=O)Nc1cc(C(F)(F)F)ccc1N1CCOCC1]

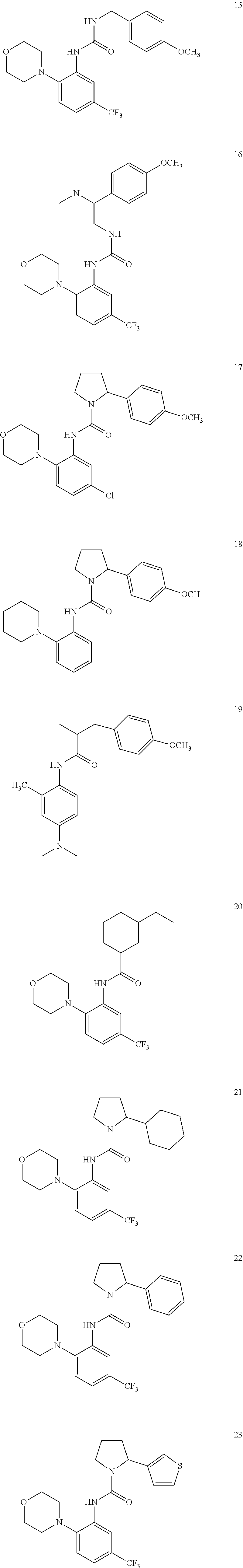

Among these molecules, showing scaffold similarity to 9, compounds 13, 16, 21, and 22 displayed increased FAK-Tyr-379 phosphorylation vs. DMSO vehicle control, while the rest of the compounds caused little change in FAK phosphorylation. Further studies performed with 13, 16, and 21, were tested in Caco-2 cells, showed that these molecules are very potent dose-dependent FAK activators at nanomolar to picomolar levels. After obtaining these encouraging results, 13, 16, and 21 to promote Caco-2 epithelial monolayer wound closure. Compared to the DMSO vehicle control, 13, 16, and 21 stimulated wound closure by approximately 10-26% over baseline wound closure rates. These results suggest that 3, 6, and 11 not only activate FAK, but also stimulate epithelial sheet migration.

Prompted by the enhancement of both FAK activation and in vitro wound closure by this set of molecules, synthesis of a small library of novel FAK activators using 13 and 16 were pursued as a basis for our further SAR studies. Physicochemical and other predicted properties of 13, 16, 21, and 22 were computed using QikProp, industry-standard Schrödinger software, for successful lead discovery The only noted potential issue is the predicted borderline hERG liability for compound 16. This liability, and possible others, could be further addressed as the series evolves with compounds with better pharmacological profile. At this point, the focus is to enhance potency for FAK phosphorylation; even though this process would not take into account the presence of chiral centers. Based on scaffold similarity among the compounds, the preliminary conclusion is that the 1-(2-morpholino-5-(trifluoromethyl)phenyl)urea moiety is essential for FAK phosphorylation. For example, when comparing FAK activation of 13 to both 18 and 19, it is clear that the lack of the morpholine ring does not enhance FAK activation.

With these results in hand, other ring moieties were modified by rational isosteric modifications. The synthetic strategy for diversified compound preparation was based on an approach to synthesizing ureas, via treatment of a phenyl carbamate, with the corresponding cyclic or aliphatic amine derivative (Scheme 1). Similarly, it was further explored if removal of the trifluoromethyl, CF₃, group is essential for FAK activation. When required, the hydrochloric salt of these analogues were pursued following standard experimental procedures.

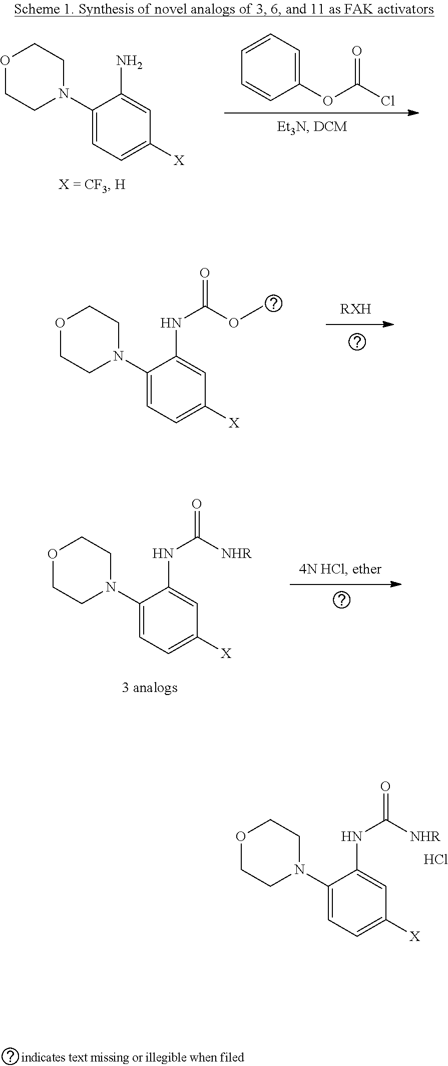

These novel compounds were tested in a biochemical assay to assess the potency to stimulate Caco-2 (by Western blotting for FAK-397) and HIEC-6 FAK activation (by a commercially available ELISA for FAK-Y-397). As seen in Table 1, on the left column, 3 and its analogs are compared in effectiveness to phosphorylate FAK measured as fold change to FAK. In this series, 13 was the most potent FAK activator. In order to enhance solubility of 13, and at the same time FAK activation, its hydrochloric salt was synthesized.

TABLE 1
Cell-based FAK-Tyr-397 activation

| | FAK-Tyr-397/FAK |
|---|---|
| Compound | fold change |
| 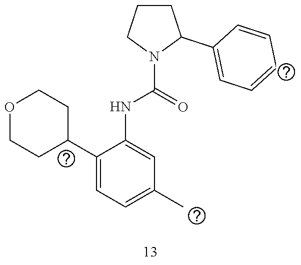 [SMILES: Cc1ccc(C2CCOCC2)c(NC(=O)N2CCCC2c2ccccc2)c1] | 1.5 |
| 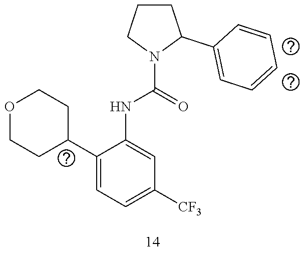 [SMILES: O=C(Nc1cc(C(F)(F)F)ccc1C1CCOCC1)N1CCCC1c1ccccc1] | 1.0 |
| 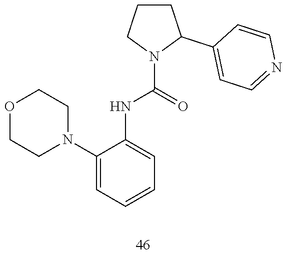 [SMILES: O=C(Nc1ccccc1N1CCOCC1)N1CCCC1c1ccncc1] | 1.1 |
| 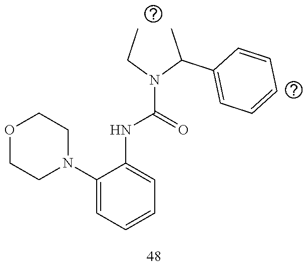 [SMILES: CCN(C(=O)Nc1ccccc1N1CCOCC1)C(C)c1ccccc1] | 1.1 |
| 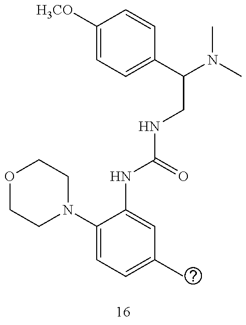 [SMILES: COc1ccc(C(CNC(=O)Nc2cc(C)ccc2N2CCOCC2)N(C)C)cc1] | 1.4 |
| 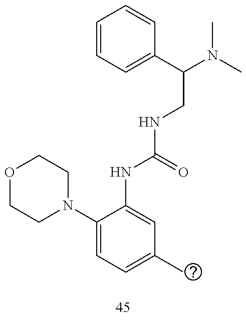 [SMILES: Cc1ccc(N2CCOCC2)c(NC(=O)NCC(c2ccccc2)N(C)C)c1] | 1.0 |
| 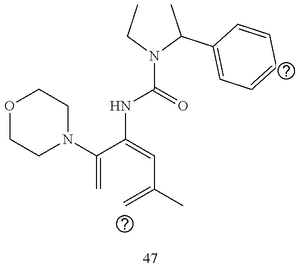 [SMILES: C=C(C)/C=C(/NC(=O)N(CC)C(C)c1ccccc1)C(=C)N1CCOCC1] | 1.4 |
| 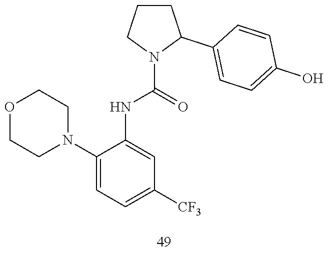 [SMILES: O=C(Nc1cc(C(F)(F)F)ccc1N1CCOCC1)N1CCCC1c1ccc(O)cc1] | 1.1 |

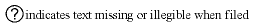

In order to enhance the solubility of 13, and at the same time facilitate treatment of cells or potentially intact organisms to induce FAK activation, its hydrochloric salt 14 was synthesized. However, 14 showed reduced activation as compared to 13. Similarly, to reduce lipophilicity, we also pursued the removal of the CF₃ moiety as seen in 46 and the synthesis of its corresponding hydrochloric salt 48. In both cases, these compounds did not induce FAK phosphorylation. The desmetoxy analog of 16, 45, was also synthesized and evaluated but did not stimulate FAK phosphorylation. Finally, 49, a 22 analog bearing a hydroxyl moiety, was pursued with the goal to increase uptake in CaCo-2 cells, by glucurodination, and thus enhance FAK phosphorylation. However, 18 also did not promote FAK phosphorylation as seen with 22.

It was originally hypothesized that by synthesizing the corresponding hydrochloric salt, reducing lipophilicity by removing the CF₃ moiety, or adding a hydroxyl group would lead to enhancement of uptake inside Caco-2 cells and thus increase FAK activation. However, the ability of a small molecule to overcome diverse issues including bioavailability, cell membrane transportation, pharmacokinetics, and others. How these various issues affect the fate of these molecules when added to cultured cells awaits investigation. Nevertheless, promising FAK activators such as 13, 16, and 21 were discovered. These molecules not only stimulate FAK phosphorylation at remarkably low concentrations, but also promote the healing of intestinal epithelial wounds in vitro. In addition, 13, 16, and 21 showed reasonable drug-like properties based on both in vitro and in silico results

### Experimental Procedures

9, 10, and the set of 11-23 screened compounds were obtained from Enamine (Monmouth Jct., NJ). All other precursors for chemical synthesis were of the highest purity available. Compounds 6 and 12 were resynthesized.

### Cell Culture

Human Caco-2 cells were from ATCC (Manassas, VA) and maintained in Dulbecco's Modified Essential Eagle's Medium supplemented with 10% FBS as described by us previously 25. 80-90% confluent Caco-2 cells were seeded into cell culture plates pacificated with 1% heat inactivated bovine serum albumin to prevent adhesion and avoid adhesion-associated background FAK activation. Suspended cells were then treated with DMSO (0.1%) or small molecules for 1 hour before harvesting for Western blotting.

### Western Blotting

Cells were lysed and protein concentration was determined via bicinchoninic acid protein assay (Thermo Fisher, Rockford, IL). The proteins were then separated by SDS-PAGE, transferred to nitrocellulose membranes, and incubated with antibodies to FAK-Tyr-397 (ab81298, 1:1000 dilution), or FAK (Anti-FAK, clone 4.47, 05-537, 1:1000 dilution EMD Millipore, Temecula, CA). Images were taken using a LICOR-Odyssey-Fc imaging system (LI-COR Biosciences, Lincoln, NE). Densitometry was conducted on exposures within the linear range, and FAK phosphorylation was calculated as the ratio of FAK-Tyr-397 intensity to FAK intensity.

### Monolayer Wound Closure Assays

Caco-2 cells were seeded at 75-80% confluence into collagen I coated 6-well plates. When the cells reached 100% confluence, wounds were made in the monolayers with non-barrier autoclaved tips. Wound images were captured using an inverted light microscope (OLYMPUS CK2, Center Valley, PA) at 0 hours and 24 hours after treatment with DMSO or small molecules. Wound areas were measured with Image J software.

### Statistical Analysis

Data are depicted graphically as mean+/−standard error, and were analyzed by t-test or ANOVA as appropriate seeking 95% confidence.

### Example 2

Aspirin and other nonsteroidal anti-inflammatory drugs (NSAIDs) have long been known to injure the gastroduodenal mucosa but are now known to injure the distal small bowel injury at even higher rates. 10-40% and 50% of chronic NSAID users develop upper or lower GI ulcers respectively. Medical costs of adverse GI events from NSAID use exceed $4 billion per year. NSAIDs injure the mucosa by two different mechanisms. Proximal GI injury reflects COX-1 inhibition. However, distal small bowel injury is caused by NSAIDs complexing to bile acids, potentiating bile acid toxicity, and by changes in the numbers and types of enteric bacteria. Proton pump inhibitors (PPIs) have been co-prescribed with NSAIDs to ameliorate proximal GI NSAID injury but they potentiate distal small bowel injury, perhaps by altering the intestinal microbiome and are no longer recommended for use in this context. Since patients depend upon NSAIDs for pain relief or cardiovascular risk reduction, there is an urgent need to find a way to treat their mucosal complications.

Surprisingly, despite studies of growth factors and cytokines, no currently available therapeutic promotes mucosal healing directly. For instance, inflammatory bowel disease therapy reduces inflammatory injury to stimulate mucosal healing. Antacids and antisecretory drugs block acid secretion to stop further injury. Healing requires epithelial cell sheet migration across the wound, often followed by proliferation to create more cells to resurface the injury. Focal adhesion kinase (FAK) is a key regulator of epithelial sheet migration and a convergent target for many growth factors. Inhibiting or reducing FAK inhibits sheet migration. However, total and activated FAK are decreased in migrating intestinal epithelial cells in vitro and in vivo at the edge of healing human ulcers. We identified 2 small molecules that mimic the FERM FAK subdomain and activate FAK at concentrations as low as 10 nM with specificity. In vivo studies with one compound, 45, demonstrated markedly accelerated healing of a standardized ischemic intestinal ulcer and then of murine NSAID-induced distal and proximal mucosal injury, without obvious toxicity. Follow-up structure-activity relationship (SAR) studies using commercially available analogs (SAR by commerce) identified several additional compounds that activate FAK at nanomolar to even picomolar concentrations. These compounds bind the FAK catalytic domain and increase its ATP-binding affinity. 40 was synthesized it is water-soluble (facilitating dosing), effective at picomolar concentrations, and exhibits no cytotoxicity at 10000× higher than effective doses. Its efficacy and non-toxicity has been verified in vivo, showing that it is enterally absorbed, and applied for patents. This compound represents a prototype therapeutic lead for a novel approach to heal NSAID-associated mucosal injury. This molecule's only drawback is a short plasma half-life, requiring suboptimal QID dosing. While this could be addressed by a sustained release formulation, a molecule with a longer half-life would be preferable. The instant disclosure identifies lead molecules with a longer half-life for further pre-clinical development as first-in-class therapeutic agents for mucosal healing.

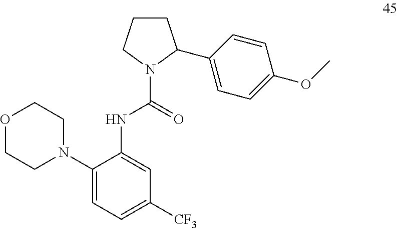 [SMILES: COc1ccc(C2CCCN2C(=O)Nc2cc(C(F)(F)F)ccc2N2CCOCC2)cc1]

Beyond over-the-counter use, NSAIDs comprise 7.7% of all prescriptions. 65% of NSAID-users develop upper or lower GI ulcers. NSAIDs injure upper GI (UGI) mucosa by COX-1 inhibition. Lower GI (LGI) injury reflects enterohepatic circulation of NSA ID-bile acid complexes that potentiate bile acid toxicity and alter the microbiome. While uncomplicated LGI erosions, like uncomplicated UGI erosions, are frequently asymptomatic, LGI (like UGI) damage can cause acute bleeding or chronic anemia, perforation, stricture, or obstruction. NSAID UGI ulcers are treated by proton pump inhibitors (PPI) or H2 blockers, but these only reduce acid without directly promoting healing. Moreover, it is known that suppressing gastric acid actually worsens NSAID small bowel enteropathy by changing the enteric microbiome. PPI are therefore no longer recommended to prevent NSAID-injury. H2 blockers are likely similar. Sucralfate co-prescription with NSAIDs reduces superficial LGI erosions but not symptoms or ulcerogenesis. Rebamipide is not FDA-approved. Misoprostol is infrequently prescribed and often poorly tolerated (8% serious side effects over 8 weeks vs. none with placebo). A 2020 review states “Misoprostol is the only drug . . . proven beneficial . . . on bleeding small intestinal ulcers induced by NSAIDs or low-dose aspirin, but its protection is insufficient.” An agent addressing UGI and LGI NSAID injury would be a major advance in avoiding and treating NSAID-induced ulcers.

Mucosal healing balances epithelial migration and proliferation to resurface injured gut against continual injury by inflammation, ischemia, and luminal agents. However, virtually all treatments only reduce injury (e.g. immunosuppressives, anti-acid agents). The only therapeutic that may promote healing is sucralfate, which some hypothesize to not only shield ulcers from injury but also bind luminal growth factors and plaster them across peptic ulcers. A therapy promoting UGI and LGI mucosal healing is an important unmet need for millions.

FAK activation is critical for mucosal healing. Healing before rebleeding would help these patients avoid surgery. Because these agents will target epithelial healing directly rather than the neutralization of ongoing injury, they might also prove useful in the future in synergistic combination with immunosuppressives for inflammatory bowel disease, which would be useful even in parenteral form for an acute IBD flare. This is mentioned only to emphasize the potential long-term significance of this work.

Aim 1: Design and Optimize the Next Generation of Lead Molecules with a Longer Half-Life, Preserving Activity and Optimizing Half-Life, Potency, Specificity, Oral Availability, and Lack of Cytotoxicity.

1.1 Background and preliminary data.

Building on 40, Aim 1 will identify preclinical drug candidates with optimized efficacy, physicochemical and ADME properties, and minimal predicted toxicity via standard medicinal chemistry strategies. 40, is active in vitro and in vim, has drug-like properties, is orally absorbed, and is neither cytotoxic nor carcinogenic. However, it has a relatively short plasma half-life. We will therefore define the metabolic or excretory mechanisms by which 40 is cleared, and design analogs predicted to resist metabolism or excretion. Analog design will be guided by biological assays differentiating compounds by potency and selectivity, by drug attributes like cytotoxicity, microsomal or hepatocyte stability, and by physicochemical properties like permeability, lipophilicity, solubility, and protein-binding. We will also consider medicinal chemistry principles such as avoiding known toxicophores and metabolically labile residues. Optimization cycles will continue until pre-clinical candidates/therapeutic leads are discovered or unexpectedly no reasonable path remains, in which case we will explore dosing 40 by sustained release technology.

FAK activation begins with a conformational change releasing the FAK kinase domain from the (inhibitory) FAK FERM domain. This frees the FAK kinase domain to autophosphorylate FAK at Tyr-397. FAK-FAK dimerization via interaction of FAT and FERM domains may also alter FAK conformation to allow FAK autophosphorylation. In either event, this initial Tyr-397 phosphorylation indicates FAK activation and invokes further conformational changes that permit further activation of FAK by other kinases such as Src.

This work began with a search to block AKT1-FAK interaction that promotes metastasis. A FAK FERM-domain-derived 7-mer amino acid peptide prevents this interaction, so it was sought to mimic this FAK subdomain's structure by in silico screening existing small molecules based on assumptions about the subdomain's tertiary conformation. A virtual screen of the ZINC Database yielded molecules based on an active FAK mutant peptide that reduced cancer cell adhesiveness. However, molecules based on the actual FAK sequence activated FAK. (Conceivably, known cancer might contraindicate treatment with such agents, but SHP2 inhibitors that activate kinases like FAK are proposed as cancer therapy.) Since FAK activation is critical for restitution, it was tested whether such molecules might not only activate FAK but also promote epithelial monolayer wound closure. The most potent were denoted as 45 and 10, selecting 45 for further study. To show that faster wound closure reflected cell motility (not proliferation), we showed that 45 stimulates wound closure even if proliferation is blocked by hydroxyurea and confirmed 45 is not mitogenic. FAK inhibition blocked 45 stimulation of wound closure suggesting 45 stimulates migration by FAK, not an off-target side effect. Indeed, 45 activates neither Pyk2, the kinase most similar to FAK, nor Src, another critical focal adhesion tyrosine kinase.

Indeed, 45 interacts directly with FAK. Adding 45 to purified full length (125 kD) human FAK and ATP stimulates ATP to ADP conversion, as well as conversion by the 35 kD FAK kinase domain. 45 also increases purified FAK-Tyr-397 autophosphorylation by Western blot. 45 increases Vₘₐₓ for ATP-ADP conversion by the 35 kD kinase domain.

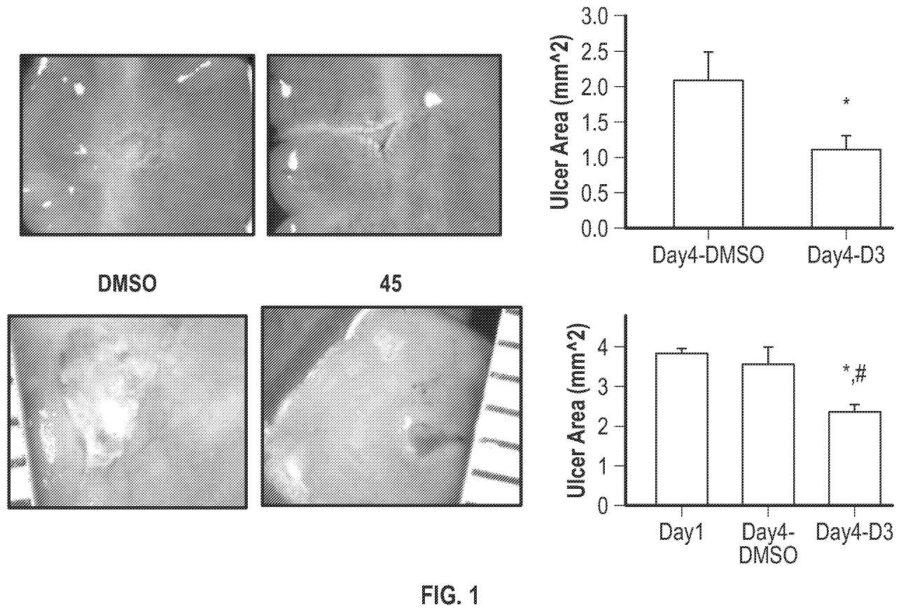

In vivo data was promising. Every 6 hours intraperitoneally, 900 ug/kg 45 markedly decreased LGI ileal ulcers in mice after indomethacin. (FIG. 1). It was confirmed that this involved stimulation of mucosal healing by demonstrating similar effects in discrete ileal ulcers created by applying acetic-acid-soaked discs to ileal serosa as we had described. 45 accelerated ulcer healing ˜4-fold.

Although no model reproducibly causes both gastric and distal intestinal NSAID injury in mice akin to humans, parallel gastric studies were performed. Mice were chronically dosed with 300 mg/kg daily aspirin without or with 45. 45 ameliorated ongoing aspirin-induced injury similarly to the PPI omeprazole (10 mg/kg). In each study, 45 was well tolerated. All mice lost weight because of the injury model, but 45-treated mice did not lose more weight, behaved normally without evident neurotoxicity, and had liver and kidney histology, ALT, and creatinine resembling vehicle controls in each model.

Based on 45, 13 structurally similar compounds were purchased with suitable in silico predicted drug-like properties. Structures and properties were predicted by industry-standard Schrödinger software. This yielded several molecules active at lower concentrations than 45. SAR analysis, including synthesizing new molecules, suggested the 1-(2-morpholino-5-(trifluoromethyl)phenyl)urea moiety connected to an aromatic ring via a short linear or cyclic hydrocarbon chain is essential but other parts of the molecule can be isosterically modified. One particularly promising molecule was 24,

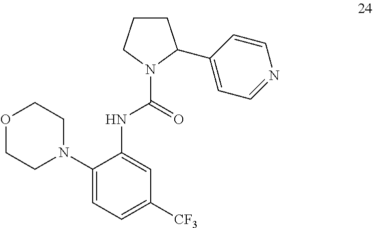 [SMILES: O=C(Nc1cc(C(F)(F)F)ccc1N1CCOCC1)N1CCCC1c1ccncc1]

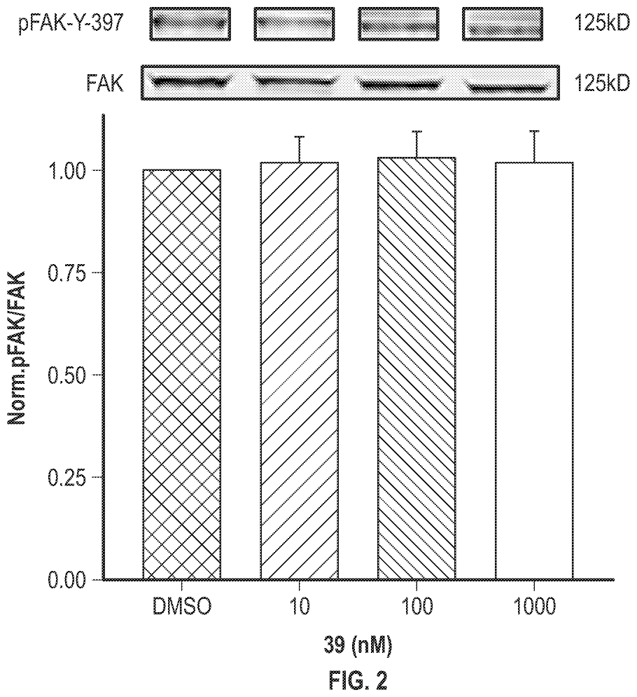

which activates FAK and stimulates in vitro wound closure at concentrations as low as 100 pM while promoting small bowel mucosal wound healing in mice over three days without obvious toxicity. However, 24, like 45 and other molecules so far studied, required DMSO solubilization with attendant DMSO toxicity. In further attempts to synthesize new molecules resembling 45 and the second-generation molecules identified in the SAR by commerce approach above, we found a new molecule, 40. 40 activates FAK in Caco-2 cells even at 100 pM, but does not activate Src or Pyk2 at 10 μM, and stimulates wound closure in vitro. Interestingly, previous tests showed its free base version, 39, to be ineffective (FIG. 2).

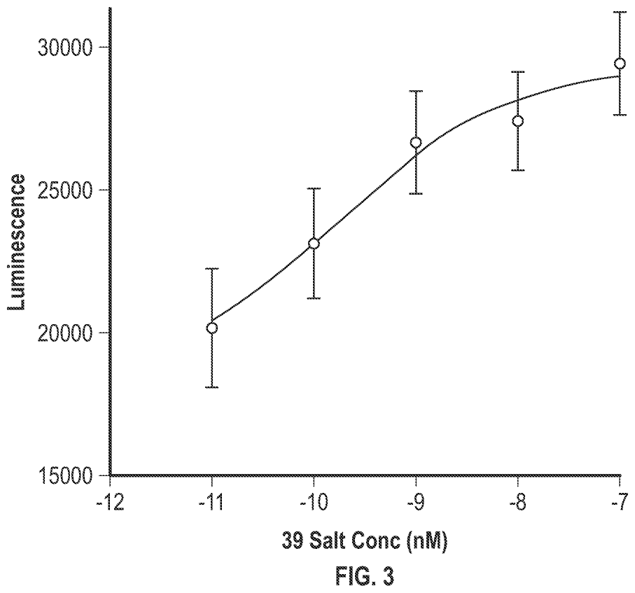

40 interacts with the FAK kinase domain and increases its Vₘₐₓ for ATP-binding (FIG. 3). The DMSO vehicle for 45 causes ˜20% cytotoxicity. Higher 45 doses are more toxic; 45 itself has an IC₅₀ of ˜50 μM. (not shown) In contrast, 40 does not require DMSO, and displays cytotoxicity at 1 mM in IMR-90 human pulmonary fibroblasts and SH-SY5Y human neuronal cells, 10⁷× higher than the 100 pM that activates FAK. 24 and 40 each have promising drug-like properties. 40 was focused on because it was not toxic at therapeutic doses and has a large therapeutic window. We studied continuous 30 mg/kg/day 40 infusion by osmotic minipump for 4 days, creating ischemic ileal mucosal ulcers by 15 second serosal application of acetic-acid-soaked discs. We saw no fur ruffling or behavioral abnormalities. Mice exhibited similar weight loss from the model (4.7±1.1% for controls with saline in the minipumps vs. 4.6±0.7% for 40-treated mice). Renal histology and plasma creatinine were normal. The model created some hepatic inflammatory changes and ALT elevation, but similarly among 40-treated mice and controls. Most importantly, 40-treated mice exhibited much smaller ulcers 4 days later.

Plasma 40 Levels at Study End were 55 nM.

24 and 40 cross the blood-brain barrier. However, no neurotoxicity was detected in vitro or in mice receiving 24 or 40, so neurotoxicity seems unlikely. Enteral gavage of 10 mg/kg of 40 yields rapid absorption, but plasma levels decrease by 2 hours. This resembles pharmacokinetics after intraperitoneal injection.

Having shown that 1.25 mg/kg/hour of 40 is effective and well-tolerated over 4 days without obvious toxicity, much higher single doses were studied to seek potential toxicity. Single intraperitoneal doses as high as 240 mg/kg seemed well tolerated, with apparently healthy mice 14 days later, although chemistries 2 days after 240 mg/kg suggested subclinical hepatotoxicity. A single dose of 480 mg/kg was lethal. One hour after 240 mg/kg dosing, plasma levels are 176 mg/ml, ˜6*10⁶× the 26 ng/ml therapeutic plasma after 4 days of 1.25 mg/kg/hr dosing. Serum chemistries at 120 mg/kg were normal (not shown).

40 is a highly promising lead. However, its short half-life could impede outpatient dosing. Aim 1 will first define the mechanisms by which 40 is metabolized and/or excreted. Following this will be design, synthesis, and thorough evaluation of the next generation of structurally optimized molecules based on our understanding of the SAR and the relevant metabolic degradation. These will be screened in molecules in vitro for FAK activation, promotion of monolayer wound closure, and cytotoxicity, and then test the most promising in vivo for stability and efficacy.

### 1.2 Overall Aim 1 Rationale.

Aim 1 will define the absorption, distribution, metabolism, and excretion (ADME) of 40 and next-generation molecules. This will permit further FAK drug optimization to achieve optimal drug-like properties including longer half-life and oral availability. The need for optimization is warranted by preliminary data indicating a short suboptimal half-life for 40. A preliminary study in mice receiving 4 days of steady-state parenteral 40 suggested much of the compound is excreted unchanged while the rest may be metabolized after parenteral administration. Since toxicity at high doses is hepatic, oral administration could yield different results such as more pronounced hepatic metabolism. In silica analysis yielded 2 structural alerts to sites that represented possible toxicity and metabolism, the morpholine ring, and the dimethylamino functional group. Furthermore, the dimethylamino functional group (sans the methyl groups) would represent an excellent site for a polyethylene glycol chain to impair excretion provided that the potency is not diminished. Therefore, an investigation of the balance between absorption, metabolism, and excretion after oral administration is warranted. Second, will be a design and synthesis of a series of molecules based on the 40 structure that optimizes ADME properties and addresses the structural alerts. These will then be screened for FAK activation, cytotoxicity, and stability in gastric and intestinal juice, and it will be investigated whether half-life has been prolonged. If not, a PEG chain will be added to the amino group moiety, or the 5-position in the phenyl ring (as a replacement for the CF; group) in case of an unexpected steric hindrance or other issues. Screening again will be carried out for FAK activity and then test successful molecules in mice for enteral absorption and more prolonged half-life, striving for an 8-hour half-life. If plasma half-life is still suboptimal, we will analyze the metabolism of our best molecules to identify possible metabolites and synthesize another generation of molecules, passing iteratively between synthesis, in vitro FAK activation screening, and pharmacokinetics until we have either prolonged the plasma half-life to 8 hours or have no further path for optimization. Since human metabolism may differ from murine metabolism, we will also assess all compounds' stability when incubated with human or murine liver cells.

### 1.3 Pharmacokinetics and Metabolomics of 40 and its Derivatives.

The new leads synthesized in 1.2 above and 1.4 below that have passed in vitro screens will first undergo an initial in vivo screening by gavaging mice at 30 mg/kg and checking plasma levels at 1, 2, 3, and 4 hours. Molecules with >4-hour plasma half-life will be assessed for longer persistence. Metabolomics/biotransformation studies will identify the major mechanisms for drug depletion from plasma to rationalize further drug modifications. We seek molecules with an 8-hour plasma half-life, but based on the above could accept 3-4 hours if necessary. Once we have acceptable plasma half-life by structural modifications, we will assess whether to move to Aim 2 or to work to further prolong half-life by identifying metabolic products of the next generation molecules and modifying them to block such degradation.

### Drug Absorption, Tissue Distribution, and Excretion Will be Analyzed by Mass Spectrometry.

Test compounds (30 mg/kg) and control vehicles will be dosed by gavage. Plasma, tissues, urine, and feces/intestine content will be collected at 0-8 hours and extracted with methanol as previously described. Targeted MS analysis will be used for intact drug pharmacokinetics. To identify products of drug biotransformations, first used will be untargeted metabolomics and then targeted MS/MS analysis to confirm and quantify these products. MS analysis will be performed. For pharmacokinetic analysis, quantification of drug concentrations by targeted MS/MS using a tandem quadrupole LC-MS/MS System (TQS, Waters) will be carried out. The following parameters will be calculated. Cₘₐₓ, Tₘₐₓ, half-life, and area under the curve (AUC). Bioavailability will be assessed by comparing AUC-plasma-p.o. versus AUC-plasma-i.v. Excretion (% of dosed intact drug excreted) with urine will be calculated as [AUCᵤᵣᵢₙₑ]/[AUCₚₗₐₛₘₐ]*100%. To assess GI excretion, we will combine intestinal contents with collected fecal samples instead of [AUCᵤᵣᵢₙₑ]. The time course for AUC calculation will be based on 7 plasma and urine samples collected through femoral vein and bladder catheters from anesthetized mice to avoid animal restraint stress.

### 1.4 Structural Optimization of 40 Analogs for Improved Plasma Stability.

Scaffold similarity among compounds suggests the 1-(2-morpholino-5-(trifluoromethyl)phenyl)-urea moiety is required for FAK phosphorylation. For example, it was found that that the removal of the morpholine ring as well as replacement of the CF₃ moiety with hydrogen does not stimulate FAK activation. It was further confirmed that the isosteric replacement of the morpholine ring with 2-oxa-6-azaspiro[3.3]heptanyl and tetrahydro-2H-pyranyl groups in both 24 and 39 ablated activity (not shown). However, replacing the CF₃ moiety with F in 24 to create 25 does not impair activity. At this point, chirality was not considered.

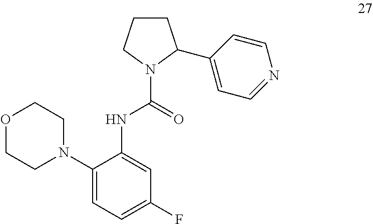 [SMILES: O=C(Nc1cc(F)ccc1N1CCOCC1)N1CCCC1c1ccncc1]

Optimization used 40 as the starting point, reserving the possibility of constructing 24 or 25 derivatives as alternatives if all 40 derivatives fail. All three compounds were subjected to computational evaluation that indicated drug-likeness, good solubility, bioavailability, and medicinal chemistry friendliness. We already know that 40 is orally absorbed. Plasma levels peak rapidly but decline by two hours. At least 20-25 new molecules were designed to impede excretion and/or metabolism and further avoid toxicity based upon our structural analysis and iterative results from the studies above.

Further syntheses will be guided by additional metabolic vulnerabilities identified by the instant metabolic studies. Standard medicinal chemistry strategies to optimize and validate identified leads by focused compound design and synthesis will be used to design new target compounds. In addition to potency and selectivity, physical properties, and rectification of ADMET liabilities by compound optimization will be critical. Lead optimization will be hypothesis-driven and informed by established medicinal chemistry methods such as investigation of SAR, bioisosteric replacements, and structural modifications (ring-breaking, ring-forming, atom replacements, linker homologation and shortening, etc.). We will incorporate functional groups and structural features that enhance solubility (ionizable groups and groups that interfere with p-stacking) when possible. Computational tools, such as Schrödinger, SwissADME, SMARTCyp (predicts sites that are most liable to Cytochrome P450 mediated metabolism), and Pipeline Pilot will guide this approach. The design will pay careful attention to clogP, clogD, and molecular weight (Lipinski's “Rule of Five”). This will increase confidence that the compounds prepared will have physical properties associated with oral bioavailability. Lead optimization will involve a closed loop of design, synthesis, purification, and assay, as above. Optimization cycles will continue until pre-clinical candidates meet the desired target lead profile (Table 4, next page) or no reasonable path forward remains.

We will synthesize >50 mg of each compound. Compounds will be ≥95% pure by UPLC/MS/PDA/ELSD with 2 solvent systems, with structures confirmed by ¹H and ¹³C NMR. See also Authentication of Key Resources.

1.4a Impeding metabolic degradation. Improvement of the metabolic stability of 40 was sought by blocking sites with potential high vulnerability to P450. Predictive computational studies with SMARTCyp suggest the 3- and 5-positions in the morpholine ring have higher reactivity. Potential formation of a highly reactive diiminoquinone moiety by liver microsomes that can lead glutathione adduct formation that could be toxic, an additional concern. Both methyl groups on the dimethylamino moiety of 40 were also flagged.

TABLE 4

| | Minimally | | | |
|---|---|---|---|---|
| | acceptable | Ideal | Compound 24 | Compound 39 |
| FAK potency | EC₅₀ < | EC₅₀ < | EC₅₀ < | HCl |
| | 10 nM in vitro | 1 nM in vitro | 10 nM in vitro | EC₅₀ < 10 nM in vitro |
| Selectivity | Pyk2, Src | Pyk2, Src | No Pyk2, Src | No Pyk2, Src |
| against relative | activation at >100 × | activation at >1000 × | activation at >1000 × | activation at >1000 × |
| off-targets | FAK | FAK | FAK | FAK |
| | activation | activation | activation | activation |
| In vitro PK | Mouse and | Mouse and | Human plasma | Human plasma |
| | human | human | stability = | stability = |
| | microsome | microsome | 98.62% at 6 h | 88.56% at 6 h |
| | stability > 50% | stability > 75% | Plasma protein | Plasma protein |
| | at 1 h | at 1 h | binding = | binding = |
| | Plasma protein | Plasma protein | 90.51% | 91.39% |
| | binding < 98% | binding < 95% | CYP 2C9 IC₅₀ = | CYP 2C9 IC₅₀ = |
| | CYP's IC₅₀ > | CYP's IC₅₀ > | 4.50 μM; | 1.25 μM; |
| | 1 μM; | 10 μM; | CYP 2C19 IC₅₀ = | CYP 2C19 IC₅₀ = |
| | Caco-2 A-B | Caco-2 A-B | 5.43 μM, CYP | 3.98 μM; CYP |
| | permeability of | permeability of | 2D6 IC₅₀ = | 2D6 IC₅₀ = |
| | 1 × 10⁻⁶ cm/s | 10 × 10⁻⁶ cm/s | 10.06 μM | 15.41 μM; |
| | with efflux | with efflux | Caco-2 | Caco-2: A-B |
| | ratio < 0.75 | ratio < 0.5 | permeability not | permeability not |
| Mouse | 30 mg/kg | ≤10 mg/kg | measured but | measured but |
| therapeutic dose | daily × | daily × 4 | readily orally | readily orally |
| In vivo PK | 4 weeks | weeks | absorbed in vivo | absorbed in vivo |
| | % F > 20 | % F > 50 | <30 mg/kg/day × | <15 mg/kg/day × |
| | CL < 30 | CL < 30 | 4 days | 4 days |
| | mL/min/kg | mL/min/kg | % F to be done | % F to be done |
| | T_{1/2} > 4 hr; | T_{1/2} > 6 hr; | T_{1/2}~1.5 hr | T_{1/2}~1.5 hr |
| | B/P ratio <1 | B/P ratio < 0.5 | B/P ratio <0.15 | B/P ratio <0.15 |
| | | | (2 h) | (2 h) |
| Physicochemical | log D_{7.4} < 4 | log D_{7.4} < 4 | log D_{7.4} = 3.12 | log D_{7.4} = 2.87 |
| properties | Solubility >10 μM | Solubility >100 μM | Solubility = | Solubility = |
| Safety | Cytotoxicity in | Cytotoxicity in | 252.41 μM | 289.29 μM |
| | IMR90 and SH- | IMR90 and SH- | Cytotoxicity in | Cytotoxicity in |
| | SY5Y cells | SYSY (LD₅₀) >1 | IMR90 and SH- | IMR90 and SH- |
| | (LD₅₀) >10 μM | mM hERG > 30 μM | SY5Y (LD₅₀) | SYSY (LD₅₀)~1 |
| | hERG > 30 μM | Mini-Ames: | >1 mM | mM |
| | Mini-Ames: | negative | hERG = 2.958 μM | hERG > 30 μM |
| | negative | In vitro safety | Mini-Ames: | Mini-Ames: |
| | In vitro safety | pharmacology: | negative | negative |
| | pharmacology: <50% | <20% for all | In vitro safety | In vitro safety |
| | for all | targets at 10 μM | pharmacology: to | pharmacology: to |
| | targets at 10 μM | Therapeutic | be done | be done |
| | Therapeutic | Index: 100 | Therapeutic | Therapeutic |
| | Index: 100 | | index: to be done | Index: ~10⁶ |

Additional evidence of the vulnerability of the dimethylamino group is that 45, which lacks that functional group, has a 2.5 h plasma half-life vs. only 1.5 h for 40. We will therefore start by targeting these sites, substituting 3- and 5-positions of the morpholine moiety with F or methyl or ethyl groups. We will also evaluate replacing the dimethylamino moiety with free amine and examine isomerization of the pyridine ring. The 5-position in the phenyl ring will also be derivatized and evaluated, if necessary. Scheme 2 presents the general synthetic strategy. We can similarly optimize the 24/25 structure if necessary.

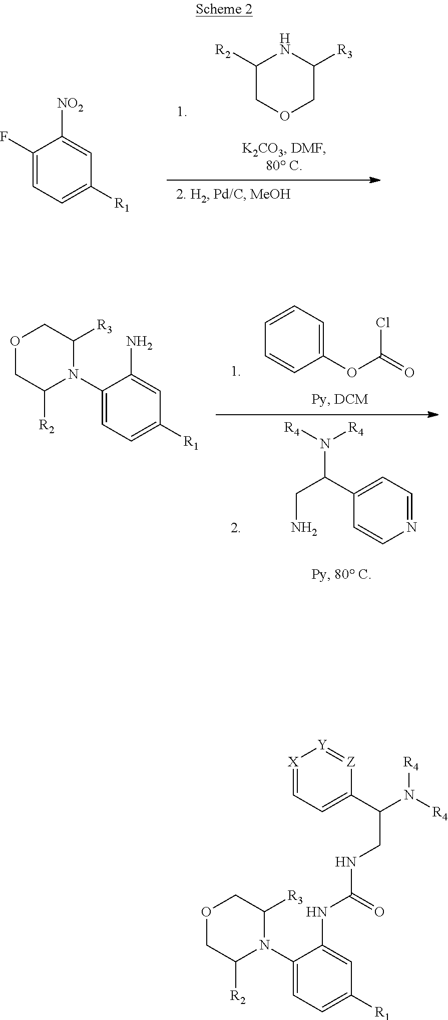

Newly synthesized molecules were screened first for their ability to activate FAK, stimulate monolayer wound closure in Caco-2 cells at 10 nM, and non-cytotoxicity at 10 μM (1000× higher) in IMR-90 and SH-SY5Y cells. Then, since Aim 1 seeks longer plasma half-life after oral dosing, we will seek molecules that resist degradation by gastric acid, pepsin, pancreatic enzymes, and bile. We will mix successful molecules with simulated human gastric and intestinal fluid, comparing by LC-MS initial concentration with concentration after 1-hour at 37° C.

The most successful molecules underwent in vitro ADMET assays, including mouse and human metabolic stability, solubility (pH 7.4), Caco-2 (permeability), and log D determination. We will evaluate lead candidates for drug-like properties, including aqueous solubility (PBS buffer, pH 7.4), pKₐ, lipophilicity, chemical stability at pH 1 (the pH of the stomach), pH 6 (small intestine), A-B partitioning (octanol-PBS buffer, pH 7.4), liver microsomal stability as an indication of potential first-pass metabolism (microsomes from mouse and humans), substrate potential for isoforms of human P450 such as CYP3A4, cell permeability (Caco-2 cells), hERG toxicity (cell lines), mutagenesis, and plasma protein binding (mouse & human). Compounds passing in vitro ADMET criteria with potency <20 nM will proceed to traditional systemic in vivo PK in mice. Computed physical properties that correlate with good oral bioavailability and cell permeability will be calculated for new compounds throughout the optimization process and will 1.5 also influence lead compound selection as will ligand efficiency (LE) and lipophilic efficiency (LLE). Molecules will be administered by gavage to mice at 30 mg/kg, and we will measure plasma levels at 2 and 4 hours. The most successful molecules in this regard (with the highest levels at 2 and 4 hours) may be further modified by developing combinations of modifications at different parts of the molecule.

1.4b. Impeding Excretion.

A parallel strategy to impede excretion will focus on polyethylene glycol (PEG) conjugation of 40 and its derivatives from 1.4a. The 5-position in the phenyl ring offers a convenient site for PEGylation by the approach in Scheme 3. We can similarly PEGylate 24,

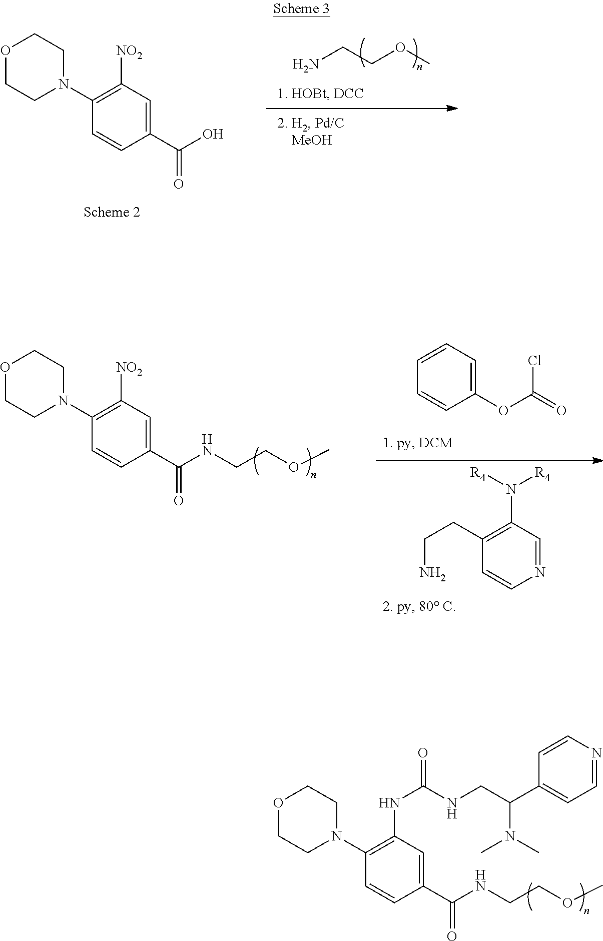

25, and their derivatives if necessary. Different chain lengths will be evaluated. Scheme 3 depicts an alternative strategy, conjugating PEG to the amino group.

Unexpected results and alternative approaches. If we unexpectedly find we cannot extend the half-life of our molecules to 8 hours, we will choose the molecules lasting longest for progression to Aim 2. We will then consider developing slow-release rate-controlled oral dosage forms such as capsules and tablets. While we focus on intermittent oral administration here, parenteral continuous infusion of such a molecule would be an appropriate and valuable therapeutic approach for acute treatment of severely ill patients, such as those with acute GI bleeding from NSAID toxicity hoping to avoid surgery. In such cases, if 3 days of parenteral treatment could tip the balance from continued illness toward recovery, the risks and complications of surgery could be avoided. Thus, even the existing 40 molecule has therapeutic potential, but we have chosen to focus on developing a molecule with a longer half-life after oral administration to broaden the potential therapeutic scope of such agents to outpatient therapy or co-administration with NSAIDs to prevent NSAID toxicity. Ibuprofen, for instance, has a half-life of 1-2 hours, is dosed over the counter at 400 mg every 4 hours, and is often prescribed at 600-800 mg every 6-8 hours for chronic pain. Thus, even an agent co-administered 3-4× daily would be practical and therapeutically valuable. Finally, preliminary studies suggest that even a modest prolongation of half-life to 2-3 hours might be useful. Wounded Caco-2 monolayers were treated with 100 nM 40 and then changed the culture medium back to medium without 40 at hours 1, 2, 3, or 4 before measuring wound closure at 24 hours, simulating 40 loss from perfusing plasma. Three-hour exposures had the same effect as 24 hours. We next compared exposure to 100 nM 40 for a full 24 hours to an initial 2-hour exposure, 10 hours in medium without the molecule, 2 more hours of exposure to the molecule, and then 10 more hours without it. All monolayers had medium changed at 2, 12, and 14 hours, without or with 40. We found equivalent stimulation of wound closure when the monolayer was exposed twice for 2 hours each to a full 24 hours of exposure, modeling twice-daily administration of a molecule that persisted for 2 hours in the plasma. Thus, our optimized molecules from aim 1 will be clinically useful even if 8-hour half-life is not achieved.

### Aim 2. Evaluate the Drug-Like Properties, Enteral Pharmacokinetics, Metabolism, Excretion, and Toxicity of the 5 Most Desirable Candidates.

Aim 1 identified molecules that stimulate epithelial sheet migration by activating FAK and have plasma half-lives of 8 hours. We will now define their properties and in vivo pharmacokinetics, using gavage to mimic oral dosing.

### 2.1 Specificity

We will assess specificity as for 45¹⁵ and 40 to confirm that the candidate molecules also do not activate Pyk2, the kinase most closely related to FAK or Src, a canonical non-receptor tyrosine kinase also localized to the focal adhesion complex. These studies will be done in human Caco-2 and HIEC6 cells.

### 2.2 Drug-Like Properties.

We will evaluate the drug-like properties of each successful candidate molecule according to the Target Product Profile (Table 4). 40 tested well (Tables 1 and 4) but modified molecules yielding unfavorable results will be discarded.

### 2.3 Pharmacokinetics.

We will administer each of the 6 most desirable candidate molecules by gavage to mice, and assess plasma levels at 4, 8, 12, 18, and 24 hours to define the full timing of the loss of plasma levels.

### 2.4 Toxicity and Single Maximally Tolerated Dose.

Recognizing that if a drug is well tolerated, the therapeutic window can also be prolonged by increasing the dose, we will seek to define toxicity and the maximally tolerated dose. We will begin by assessing the single-dose maximally tolerated oral dose (MTD), the highest dose in which the mouse's clinical condition is maintained. Several groups suggest 2 mice/dose level with a doubling dose-escalation or halving dose de-escalation design. This agrees with guidelines to reduce and refine experimental mouse numbers. MTD testing will start at 10 mg/kg gavage. If mice are well after 4 days, we will double the dose.

### 2.5 Toxicity of Repetitive Dosing Over Time.

We will choose a dosing schedule (1-4×/day) based on the plasma half-life of each of the 5 most successful molecules from the above tests and administer the molecule by gavage to four mice (two male and two female) each over 4 weeks at the MTD, observing behavior and weight changes, measuring clinical serum chemistries as in Table 3, and studying organ histology as above. If mice die or demonstrate chemical or histological toxicity, we will halve the dose and repeat the study until we define the highest dose that can be administered chronically over 4 weeks without toxicity. At the sacrifice of this final series of mice, we will also assess tissue distribution of the candidate molecule in each of the tissues listed above and will assay urinary and fecal levels of each molecule and its previously defined metabolites at the time of sacrifice.

Unexpected results and alternative approaches: Since 40 seems well-tolerated at much higher doses than efficacy requires, we do not expect our next-generation molecules to be highly toxic. A narrow therapeutic window for a candidate molecule would drop it from study. If we unexpectedly cannot define a wide therapeutic window for any new molecule, we will either return to Aim 1, synthesizing new molecules that might be safer or proceed to Aim 3 with 40 itself, employing compositional drug delivery modifications such as time-release capsules or excipients to prolong its orally administered half-life.

Aim 3. Show that at Least 2 Molecules Heal NSAID-Induced UGI and LGI Injury with Minimal Toxicity.

General approach: Murine NSAID injury models are also heterogeneous, requiring substantial animal numbers for statistical significance, and risk the criticism that drugs might interfere with NSAID absorption or metabolism. We will therefore adopt a three-part approach. First, we will test candidates for the ability to heal ischemic mucosal ileal ulceration. This is much less variable, reducing mouse numbers for screening, and also shows that drug effects truly reflect promotion of mucosal healing and not interference with NSA ID mechanisms. Dose-response studies in this model will also require fewer mice. Second, we will take the maximally effective and tolerated dose from this first model and confirm results in models of UGI and LGI NSAID injury.

### 3.1. Ischemic Ulcer Model Mucosal Healing.

We have previously used serosal application of a disk soaked in dilute acetic acid to create well-demarcated reproducible mucosal small bowel ulcerations without opening the bowel, and reported that healing of such wounds is accelerated by 45 and 24. An acceleration of healing by 40 was demonstrated. We will create ulcers, allow 24 hours for recovery, return of GI function, and ulcer formation, and then administer candidate molecules by gavage at a maximally tolerated dose and time interval based on Aim 2 results. Although we have not observed sex differences previously, we will study male and female mice, pooling results if we find no differences. We will monitor mouse weight and behavior until sacrifice at day 4 when the ulcer area will be measured as our primary endpoint. We will also assay plasma drug levels to confirm that absorption has not been affected and assess plasma levels of any markers of potential toxicity identified in Aim 2. We will also save tissue from any organs identified as sites of toxicity in Aim 2 to continue to seek evidence of toxicity in these sicker animals. Ulcer tissue will be sectioned for histologic evaluation of edema and inflammatory infiltration and stained for pFAK to confirm activation in the target tissue.

We will then choose the 5 most successful candidate molecules for dose-response studies based on the maximal acceleration of healing and a wide therapeutic index, repeating the study at 75%, 50%, and 25% of the maximal dose used above to explore dose-responsiveness. We will not study higher doses as Aim 2 showed them toxic.

### 3.2. Indomethacin Small Bowel Ulcer Healing.

We will confirm the most successful 3 molecules' results using indomethacin to injure the small bowel, as we have done for 45. Although the ischemic ulcer model in 3.1 offers more reproducible quantitation of a focal ulcer area than the multiple irregular ulcers produced by NSAIDs, indomethacin injury will validate clinical relevance to NSAID-LGI toxicity. We will inject 15 mg/kg indomethacin subcutaneously, which reproducibly produces diffuse small bowel injury, and treat with vehicle or drug over 4 days as in 3A or misoprostol 800 ug/kg daily or both together. Ulcer area will be calculated after sacrifice by two blinded observers from photographs of the entire small bowel to ensure rigor and reproducibility, and histologic analysis will assess edema, inflammation, and hyperemia. We will monitor mouse weight and activity daily and save serum and tissues for toxicology as indicated. 45 substantially and statistically significantly 1.5 reduced ulcer area and provided data for us to estimate the required sample size for these studies at 17/group. Although the trend did not achieve statistical significance, 45-treated mice lost less weight in this model, as in the acetic acid model, suggesting improved well-being related to 45 effects on healing and minimal gross toxicity. Renal and hepatic histology was unchanged. Creatinine was normal and minor ALT elevation in both groups resembled that from the ethanol vehicle for indomethacin.

3.3. Healing of Gastric Mucosal Injury from Chronic Aspirin Use.

Finally, because upper and lower GI injury have different mechanisms and we must treat both, and because the ongoing injury is harder to treat than an acute insult, we will confirm that the molecules that heal distal small bowel indomethacin injury in 3B can also treat chronic gastric injury when co-administered with aspirin. Mice will receive 300 mg/kg aspirin suspended in 1% Tween 80 by gavage daily for 5 days and either vehicle or test agent (17 mice/group) by gavage at appropriate intervals starting one day later. At sacrifice on day 6, blood will be collected by cardiac puncture to assay drug levels and ALT and creatinine. The stomach, kidney, liver, heart, and lung will be harvested for morphometric and histological analysis. The stomach will be opened, photographed for blinded assessment of gross mucosal damage, and fixed for histology to quantitate the % affected area with hyperemia and inflammatory cells in the mucosa, submucosa, and muscularis, and the thickness of each layer by blinded observers as described.

Preliminary work shows 45 substantially ameliorates ongoing chronic gastric aspirin injury in mice, comparably to omeprazole. This further suggests that the lead compound will develop and can treat both UGI and LGI injury from NSAID's, even if NSAIDs cannot be stopped. The lead, 40, is a potent nanomolar in vivo activator without obvious toxicity. It is water-soluble, orally absorbed, well-tolerated both over four days or after a single dose several orders of magnitude higher than required for efficacy. It is potent and drug-like but has the single shortcoming of a short plasma half-life. These studies will further modify 40 to optimize its plasma half-life while preserving its specificity, oral bioavailability, efficacy, and drug-like properties, and avoiding toxicity. By the end of this work, we will validate at least two compounds as preclinical therapeutic leads to promote GI mucosal healing in general and after NSAID or aspirin injury in particular, demonstrating their potency, selectivity, and efficacy in three different models of mucosal injury. Future work will advance these molecules to IND-enabling animal studies and human trials. This work will therefore help develop first-in-class novel therapeutics of considerable clinical relevance.

### Example 3

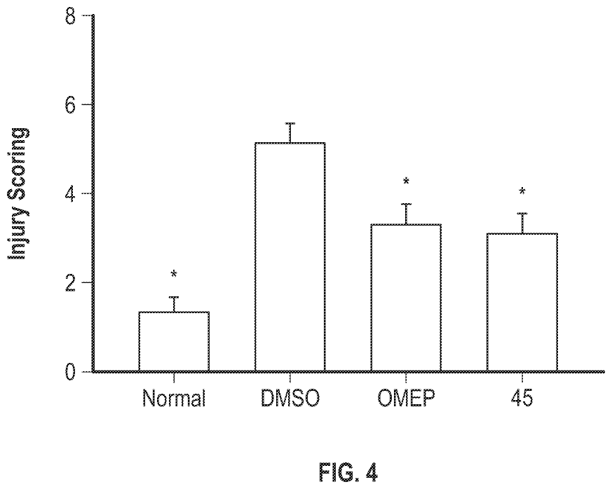

45 was further shown to promote healing in the stomach in a chronic aspirin injury model. FIG. 4 demonstrates typical gross and microscopic appearance of normal (NL) gastric mucosa, the mucosa of mice injured by chronic exposure to aspirin and receiving only the DMSO vehicle (DMSO), the mucosa of mice injured by aspirin and receiving concurrent omeprazole (OMEP) which is the conventional therapy currently available, and the mucosa of mice injured by aspirin and receiving concurrent 45 (45).

It was demonstrated that 45 acts directly on FAK in an in vitro kinase assay. This is an important advance because it establishes that the mechanism of action is directly on FAK and not via some other intermediary in the cell. We initially did these studies with the full length 125 kDa FAK molecule, and then performed further studies that demonstrated that in fact the 45 directly interacts with and activates the 35 kD kinase domain of the FAK molecule.

Another compound, termed 24, has now been tested in vitro and in vivo. In vitro, we have shown that it works at a considerably lower concentration than 45. Incidentally, although most of these molecules are water-insoluble, requiring a DMSO vehicle for solubilization, which impedes high concentration dosing both in vitro and in vivo, we have subjectively observed that 24 is somewhat less insoluble. It still requires DMSO, but less so. We have further demonstrated in vivo that 24 can heal intestinal mucosal ulcers in our ischemic ulcer model. It should be noted for IP purposes that 24 is a purchased molecule, although there is no known or described function of 24 to our knowledge and it has never been described before to either activate FAK or promote mucosal healing.

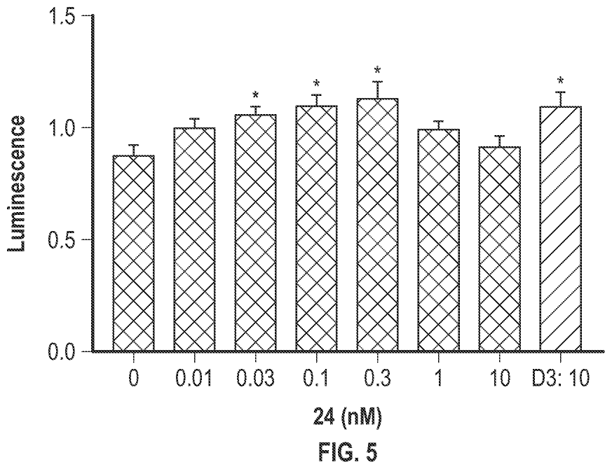

The dose-responsive effect of 24 in the in vitro kinase assay, mixing with purified protein of the FAK 35 kD kinase domain is shown in FIG. 5. 45 at 10 nM is shown for comparison as a positive control Note that 24 achieves maximal effect at about 0.3 nM, whereas 45 requires 10 nM to have a reliable effect, so that it appears 24 is effective at a lower concentration. It was also found that effective concentrations here appear lower than effective concentrations in intact cells, presumably because the cell is a more complex system and because not all added molecule gets to the target site in the cell.

The structure of 24 can be seen in the below, along with that of a number of other compounds tested, of which 24, 31, 36, and 37 seemed to activate FAK.

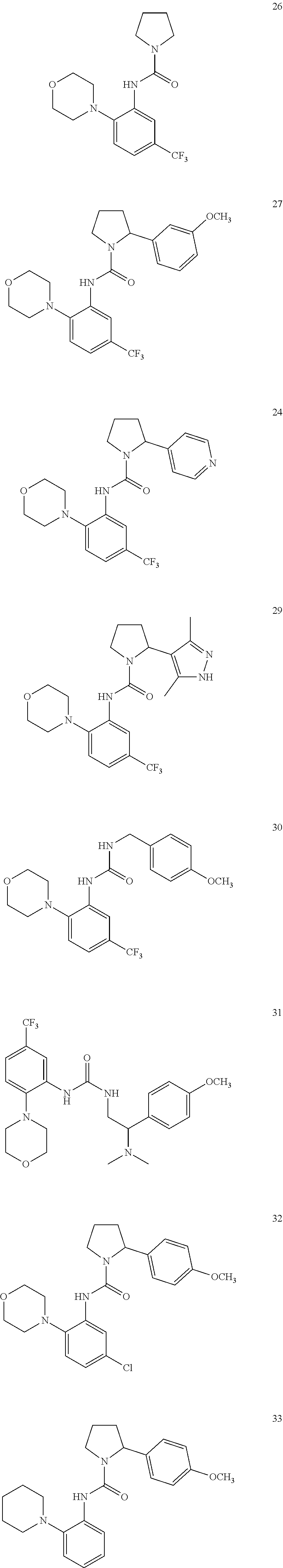

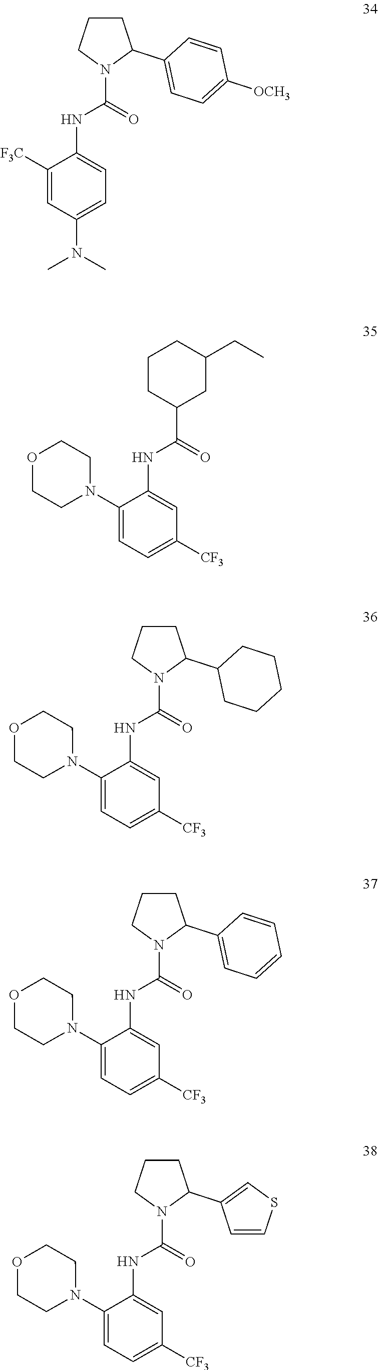 [SMILES: COc1ccc(C2CCCN2C(=O)Nc2ccc(N(C)C)cc2C(F)(F)F)cc1 ; CCC1CCCC(C(=O)Nc2cc(C(F)(F)F)ccc2N2CCOCC2)C1 ; O=C(Nc1cc(C(F)(F)F)ccc1N1CCOCC1)N1CCCC1C1CCCCC1 ; O=C(Nc1cc(C(F)(F)F)ccc1N1CCOCC1)N1CCCC1c1ccccc1 ; O=C(Nc1cc(C(F)(F)F)ccc1N1CCOCC1)N1CCCC1c1ccsc1]

The compound 39 was shown to have major advantages. For example, 40 is truly water soluble and does not require DMSO solubilization at all. This represents a major advance from the perspectives of dosing and administration. We have shown that 40 activates FAK directly in our purified in vitro kinase assay, activates FAK in intact cells in culture, and promotes epithelial monolayer wound healing in cell culture. Tests of 40 in vivo are expected to be yield results by the end of the calendar year.

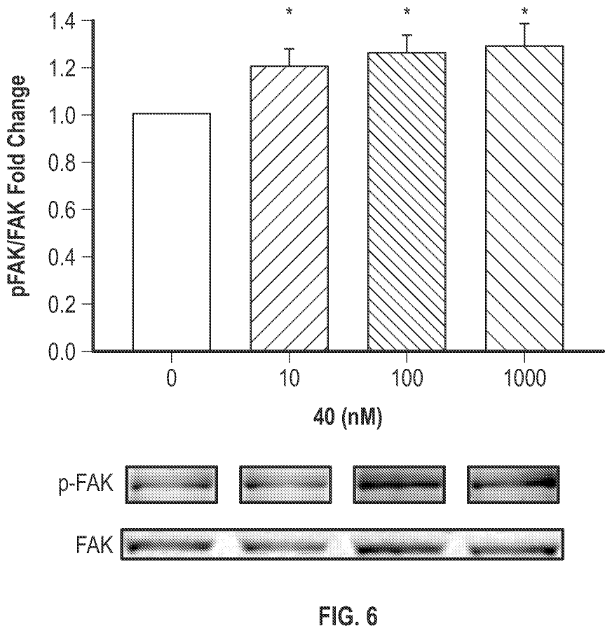

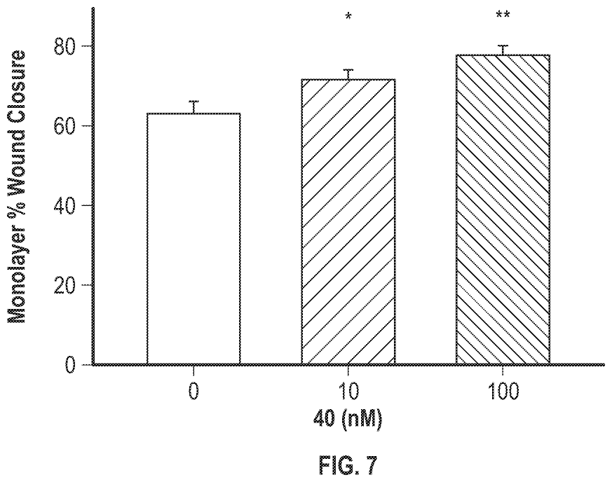

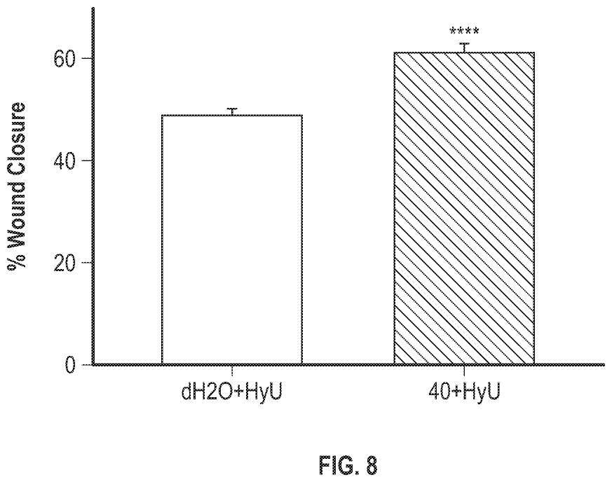

FIG. 6 demonstrates that 40 activates FAK within Caco-2 cells as assessed by Western blot for FAK-Y-397 phosphorylation. FIG. 7 demonstrates a dose-responsive stimulation of Caco-2 monolayer wound closure in vitro by 40. FIG. 8 confirms that 10 nM of 40 still stimulates Caco-2 monolayer wound closure even in the presence of hydroxyurea to prevent cell proliferation.

The structure of 40 can be seen in the below along with some other molecules that were synthesized. 40 was the most successful.

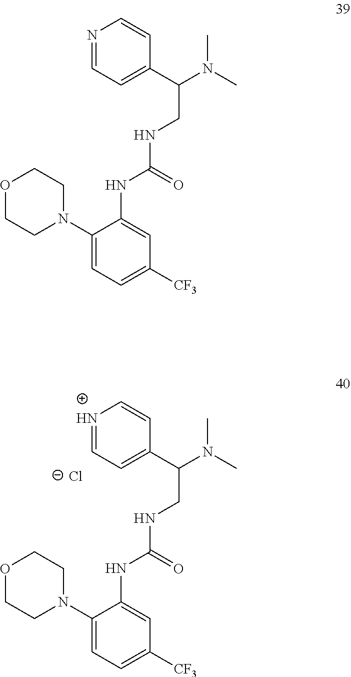 [SMILES: CN(C)C(CNC(=O)Nc1cc(C(F)(F)F)ccc1N1CCOCC1)c1ccncc1 ; CN(C)C(CNC(=O)Nc1cc(C(F)(F)F)ccc1N1CCOCC1)c1cc[nH+]cc1 ; [Cl-]]

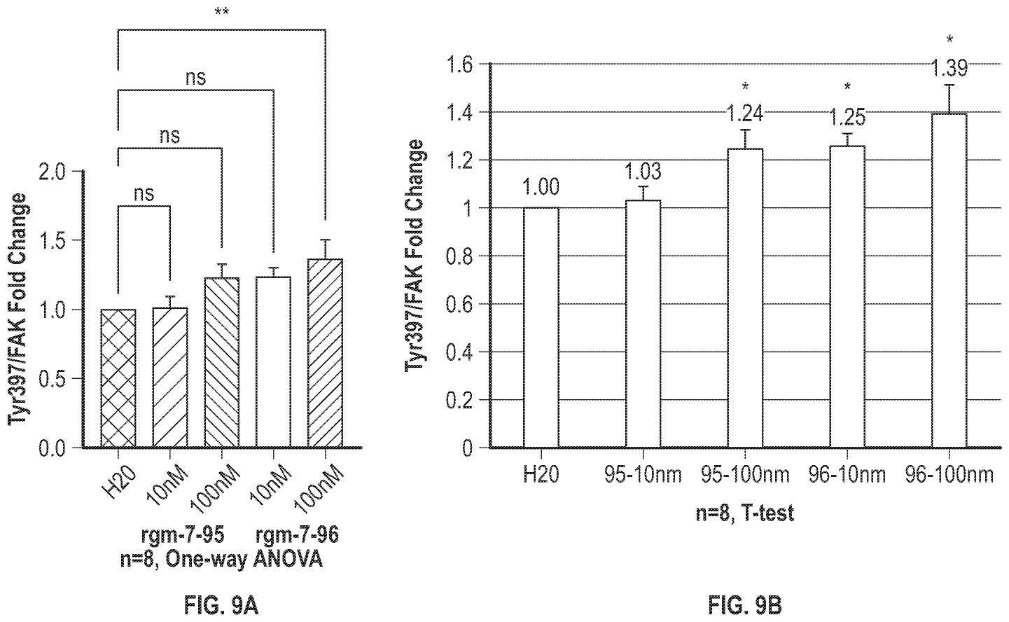

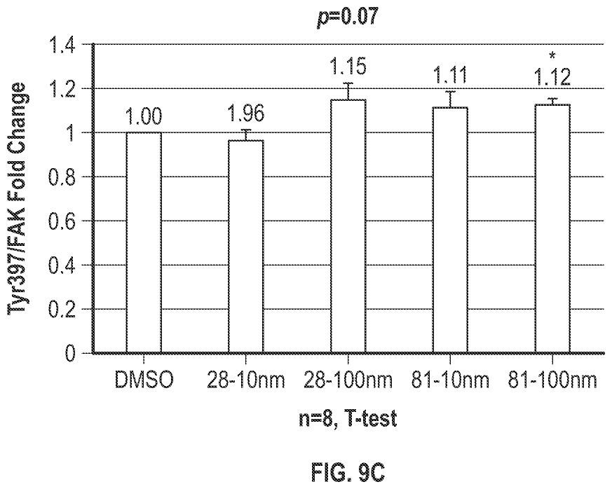

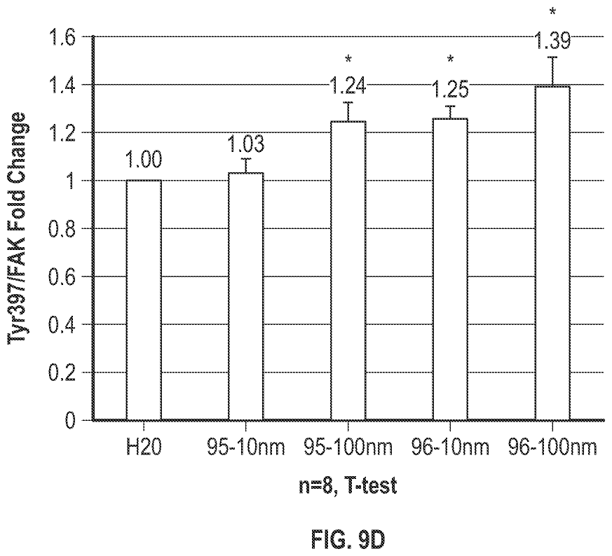

Additional molecules 41, 42, 43, and 44 were synthetized and tested. The corresponding structures are shown below. FIGS. 9A and 9B show the effectiveness of 43 and 42 at various concentrations. FIGS. 9C and 9D show the effectiveness of 43 and 42 at various concentrations.

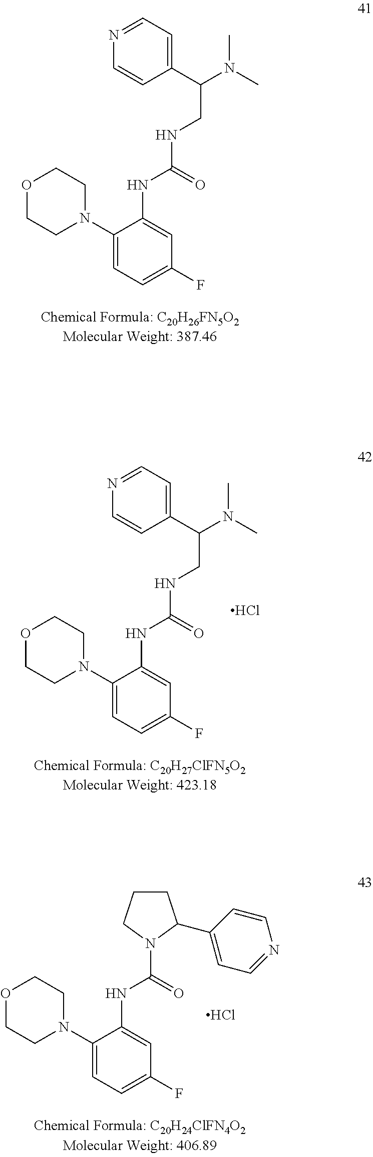 [SMILES: CN(C)C(CNC(=O)Nc1cc(F)ccc1N1CCOCC1)c1ccncc1 ; CN(C)C(CNC(=O)Nc1cc(F)ccc1N1CCOCC1)c1ccncc1 ; Cl ; O=C(Nc1cc(F)ccc1N1CCOCC1)N1CCCC1c1ccncc1 ; Cl]

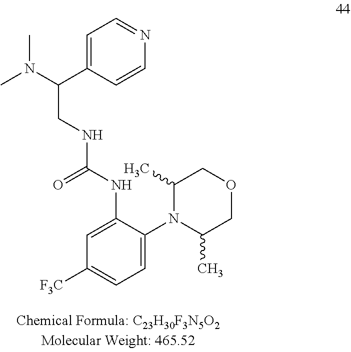

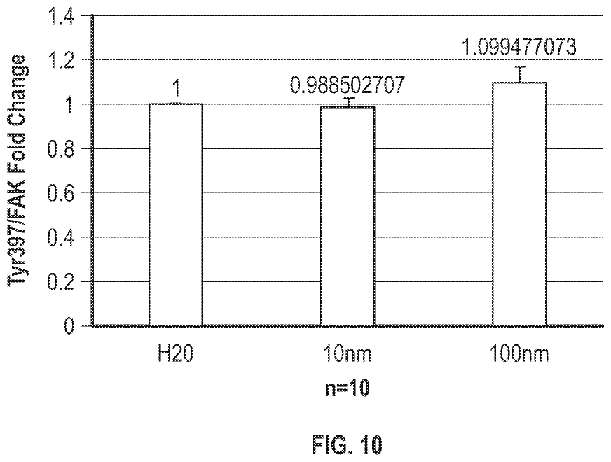

An additional molecule 45 was synthesized. 45 is the salt version of 44. The structure of 45 is presented below. FIG. 10 shows the effectiveness of 45 at various concentrations.

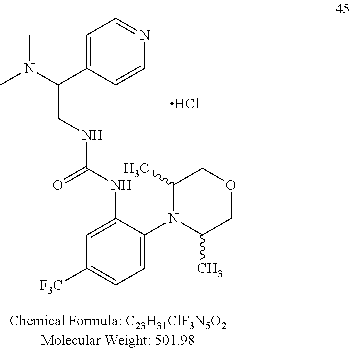

All publications, patents and patent applications are incorporated herein by reference. While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

### Exemplary Aspects

The following exemplary aspects are provided, the numbering of which is not to be construed as designating levels of importance: Aspect 1 provides a molecule used to treat an epithelial disease comprising a compound of Formula I or a salt thereof,

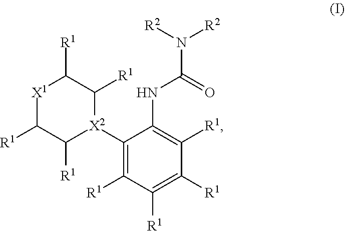

wherein
   X¹ is —C—, —N—, or —O—;
X² is —N— or —C—; and
   R¹ and R² are independently selected from the group consisting of —H, —OH, and substituted or unsubstituted (C₁-C₂₀)hydrocarbyl, and combinations thereof.

Aspect 2 provides the molecule of Aspect 1, wherein the (C₁-C₂₀)hydrocarbyl is selected from the group consisting of (C₁-C₂₀)alkyl, (C₁-C₂₀)alkenyl, (C₁-C₂₀)alkynyl, (C₁-C₂₀)acyl, (C₁-C₂₀)cycloalkyl, (C₁-C₂₀)aryl, (C₁-C₂₀)alkoxy, (C₁-C₂₀)haloalkyl, and combinations thereof.

Aspect 3 provides the molecule of Aspect 1, wherein the compound is represented by Formula II:

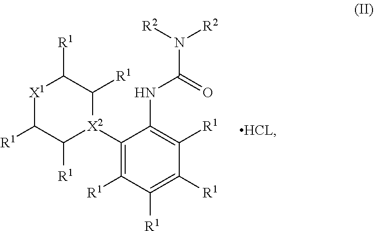

wherein
   X¹ is —C—, —N—, or —O—;
X² is —N— or —C—; and
   R¹ and R² are independently selected from the group consisting of —H, —F, —OH, substituted or unsubstituted (C₁-C₂₀) hydrocarbyl, and combinations thereof.

Aspect 4 provides the molecule of Aspect 3, wherein the (C₁-C₂₀)hydrocarbyl is selected from the group consisting of (C₁-C₂₀)alkyl, (C₁-C₂₀)alkenyl, (C₁-C₂₀)alkynyl, (C₁-C₂₀)acyl, (C₁-C₂₀)cycloalkyl, (C₁-C₂₀)aryl, (C₁-C₂₀)alkoxy, (C₁-C₂₀)haloalkyl, and combinations thereof.

Aspect 5 provides the molecule of Aspect 1, wherein the compound is represented by Formula III:

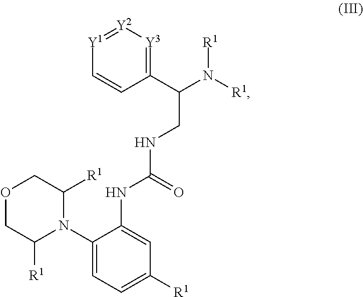

wherein
at each occurrence, R¹ is independently selected from the group consisting of —H, —F, —CF₃, —CH₃, —CH₂CH₃, —OCH₃, —OCH₂CH₃, and combinations thereof, and
Y¹, Y², and Y³ are independently selected from the group consisting of —C—, —N—, —O—, and combinations thereof.

Aspect 6 provides the molecule of Aspect 1, wherein the compound is represented by Formula N:

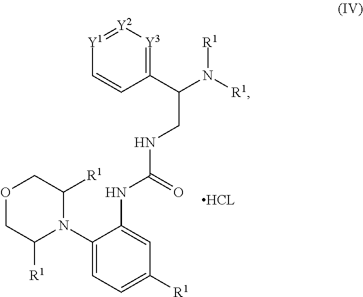

Wherein at each occurrence, R¹ is independently selected from the group consisting of —H, —F, —CF₃, —CH₃, —CH₂CH₃, —OCH₃, —OCH₂CH₃, and combinations thereof; and
Y¹, Y², and Y³ are independently selected from the group consisting of —N—, —C—, —O—, and combinations thereof.

Aspect 7 provides the molecule of Aspect 1, wherein the compound is represented by Formula V:

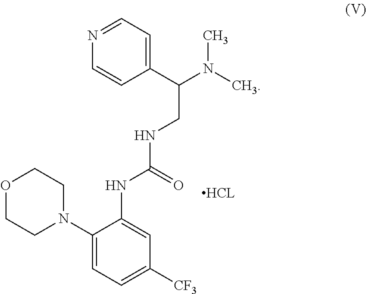

Aspect 8 provides the molecule of Aspect 1, wherein the compound is represented by Formula VI:

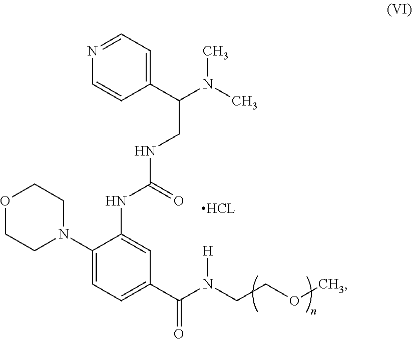

wherein n is any positive integer.

Aspect 9 provides the molecule of Aspect 1, wherein the compound is represented by Formula VII:

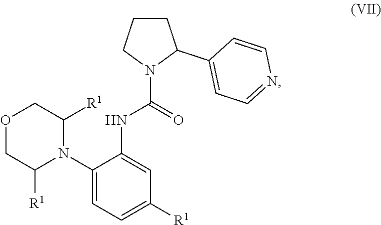

wherein at each occurrence, R¹ is independently selected from the group consisting of —H, —F, —CF₃, —CH₃, —CH₂CH₃, —OCH₃, —OCH₂CH₃, and combinations thereof.

Aspect 10 provides the molecule of Aspect 1, wherein the compound is represented by Formula VIII:

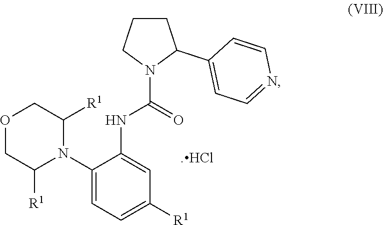

wherein at each occurrence, R¹ is independently selected from the group consisting of —H, —F, —CF₃, —CH₃, —CH₂CH₃, —OCH₃, —OCH₂CH₃, and combinations thereof.

Aspect 11 provides a method to treat an epithelial disease comprising administering to a subject in need thereof an effective amount of a compound of Formula I or a salt thereof,

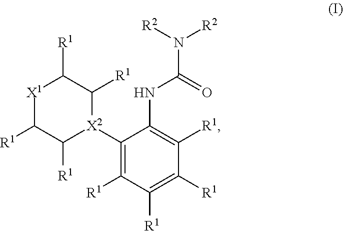

wherein
   X¹ is —C—, —N—, or —O—;
X² is —N— or —C—; and
   R¹ and R² are independently selected from the group consisting of —H, —OH, and substituted or unsubstituted (C₁-C₂₀)hydrocarbyl, and combinations thereof.

Aspect 12 provides the method of Aspect 11, wherein the (C₁-C₂₀)hydrocarbyl is selected from the group consisting of (C₁-C₂₀)alkyl, (C₁-C₂₀)alkenyl, (C₁-C₂₀)alkynyl, (C₁-C₂₀)acyl, (C₁-C₂₀)cycloalkyl, (C₁-C₂₀)aryl, (C₁-C₂₀)alkoxy, (C₁-C₂₀)haloalkyl, and combinations thereof.

Aspect 13 provides the method of Aspect 11, wherein the compound is represented by Formula II:

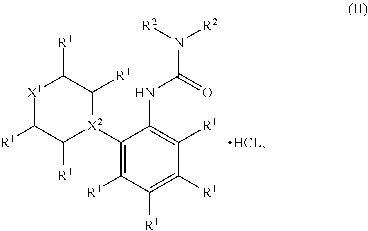

wherein
   X¹ is —C—, —N—, or —O—;
X² is —N— or —C—; and
   R¹ and R² are independently selected from the group consisting of —H, —F, —OH, substituted or unsubstituted (C₁-C₂₀) hydrocarbyl, and combinations thereof.

Aspect 14 provides the method of Aspect 13, wherein the (C₁-C₂₀)hydrocarbyl is selected from the group consisting of (C₁-C₂₀)alkyl, (C₁-C₂₀)alkenyl, (C₁-C₂₀)alkynyl, (C₁-C₂₀)acyl, (C₁-C₂₀)cycloalkyl, (C₁-C₂₀)aryl, (C₁-C₂₀)alkoxy, (C₁-C₂₀)haloalkyl, and combinations thereof.

Aspect 15 provides the method of Aspect 11, wherein the compound is represented by Formula III.

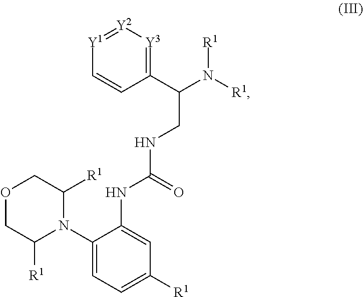

wherein
at each occurrence, R¹ is independently selected from the group consisting of —H, —F, —CF₃, —CH₃, —CH₂CH₃, —OCH₃, —OCH₂CH₃, and combinations thereof; and
Y¹, Y², and Y³ are independently selected from the group consisting of —C—, —N—, —O—, and combinations thereof.

Aspect 16 provides the method of Aspect 11, wherein the compound is represented by Formula IV:
wherein the compound is represented by Formula IV:

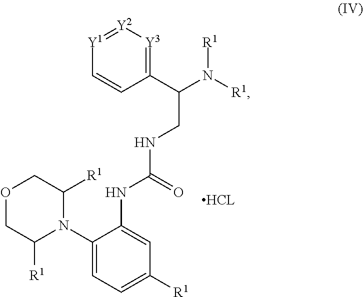

wherein
at each occurrence, R¹ is independently selected from the group consisting of —H, —F, —CF₃, —CH₃, —CH₂CH₃, —OCH₃, —OCH₂CH₃, and combinations thereof; and
Y¹, Y², and Y³ are independently selected from the group consisting of —N—, —C—, —O—, and combinations thereof.

Aspect 17 provides the method of Aspect 11, wherein the compound is represented by Formula V:

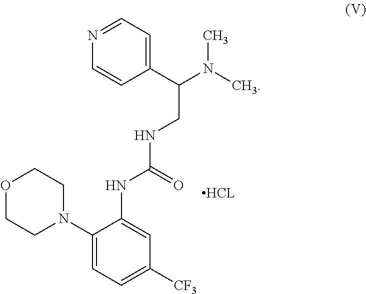

Aspect 18 provides the method of Aspect 11, wherein the compound is represented by Formula VI:

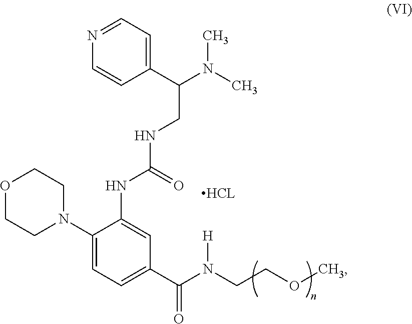

wherein n is any positive integer.

Aspect 19 provides the method of Aspect 11, wherein the compound is represented by Formula VII:

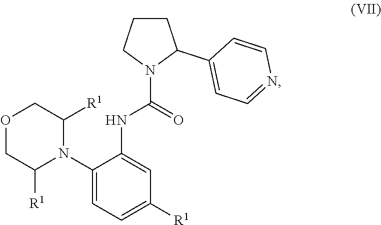

wherein at each occurrence, R¹ is independently selected from the group consisting of —H, —F, —CF₃, —CH₃, —CH₂CH₃, —OCH₃, —OCH₂CH₃, and combinations thereof.

Aspect 20 provides the method of Aspect 11, wherein the compound is represented by Formula VIII:

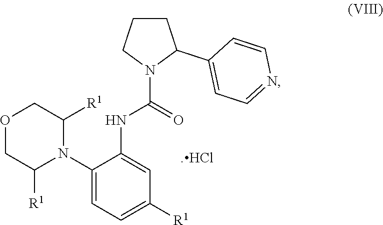

wherein at each occurrence, R¹ is independently selected from the group consisting of —H, —F, —CF₃, —CH₃, —CH₂CH₃, —OCH₃, —OCH₂CH₃, and combinations thereof.

What is claimed is:

1. A molecule used to treat an epithelial disease comprising a compound of Formula I or a salt thereof, 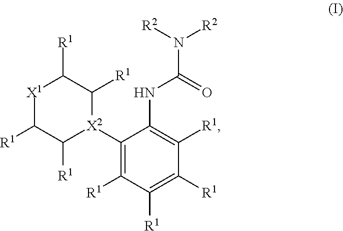
wherein
X¹ is —C—, —N—, or —O—;
X² is —N— or —C—; and
R¹ and R² are independently selected from the group consisting of —H, —OH, and substituted or unsubstituted (C₁-C₂₀)hydrocarbyl, and combinations thereof.

2. The molecule of claim 1, wherein the (C₁-C₂₀)hydrocarbyl is selected from the group consisting of (C₁-C₂₀)alkyl, (C₁-C₂₀)alkenyl, (C₁-C₂₀)alkynyl, (C₁-C₂₀)acyl, (C₁-C₂₀)cycloalkyl, (C₁-C₂₀)aryl, (C₁-C₂₀)alkoxy, (C₁-C₂₀)haloalkyl, and combinations thereof.

3. The molecule of claim 1, wherein the compound is represented by Formula H: 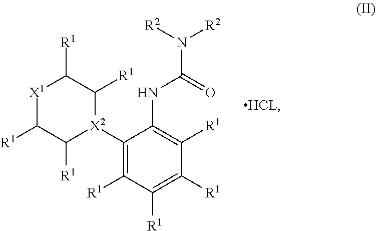
wherein
X¹ is —C—, —N—, or —O—;
X² is —N— or —C—; and
R¹ and R² are independently selected from the group consisting of —H, —F, —OH, substituted or unsubstituted (C₁-C₂₀) hydrocarbyl, and combinations thereof.

4. The molecule of claim 3, wherein the (C₁-C₂₀)hydrocarbyl is selected from the group consisting of (C₁-C₂₀)alkyl, (C₁-C₂₀)alkenyl, (C₁-C₂₀)alkynyl, (C₁-C₂₀)acyl, (C₁-C₂₀)cycloalkyl, (C₁-C₂₀)aryl, (C₁-C₂₀)alkoxy, (C₁-C₂₀)haloalkyl, and combinations thereof.

5. The molecule of claim 1, wherein the compound is represented by Formula III: 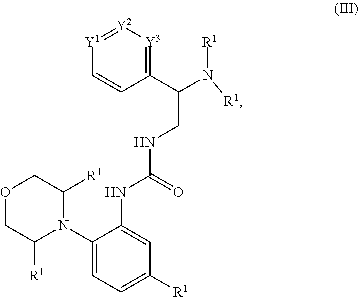
wherein
at each occurrence, R¹ is independently selected from the group consisting of —H, —F, —CF₃, —CH₃, —CH₂CH₃, —OCH₃, —OCH₂CH₃, and combinations thereof; and
Y¹, Y², and Y³ are independently selected from the group consisting of —C—, —N—, —O—, and combinations thereof.

6. The molecule of claim 1, wherein the compound is represented by Formula IV: 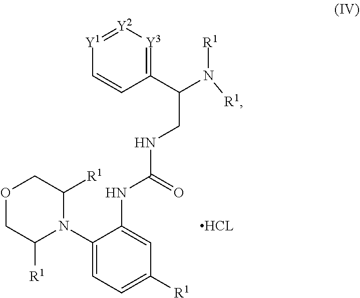
Wherein at each occurrence, R¹ is independently selected from the group consisting of H, —F, —CF₃, —CH₃, —CH₂CH₃, —OCH₃, —OCH₂CH₃, and combinations thereof; and
Y¹, Y², and Y³ are independently selected from the group consisting of —N—, —C—, —O—, and combinations thereof.

7. The molecule of claim 1, wherein the compound is represented by Formula V:

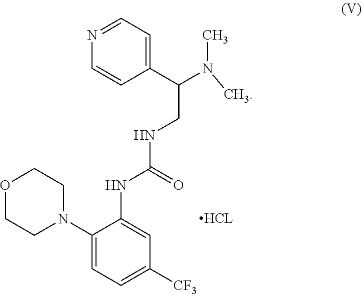

8. The molecule of claim 1, wherein the compound is represented by Formula VI: 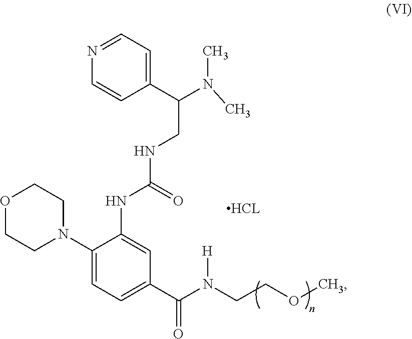
wherein n is any positive integer.

9. The molecule of claim 1, wherein the compound is represented by Formula VII: 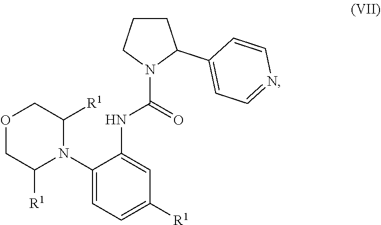
wherein at each occurrence, R¹ is independently selected from the group consisting of —H, —F, —CF₃, —CH₃, —CH₂CH₃, —OCH₃, —OCH₂CH₃, and combinations thereof.

10. The molecule of claim 1, wherein the compound is represented by Formula VIII: 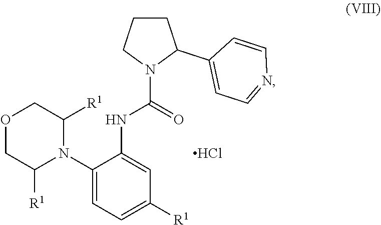
wherein at each occurrence, R¹ is independently selected from the group consisting of —H, —F, —CF₃, —CH₃, —CH₂CH₃, —OCH₃, —OCH₂CH₃, and combinations thereof.

11. A method to treat an epithelial disease comprising administering to a subject in need thereof an effective amount of a compound of Formula I or a salt thereof, 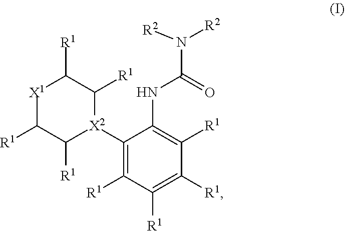
wherein
X¹ is —C—, —N—, or —O—;
X² is —N— or —C—; and
R¹ and R² are independently selected from the group consisting of —H, —OH, and substituted or unsubstituted (C₁-C₂₀)hydrocarbyl, and combinations thereof.

12. The method of claim 11, wherein the (C₁-C₂₀)hydrocarbyl is selected from the group consisting of (C₁-C₂₀)alkyl, (C₁-C₂₀)alkenyl, (C₁-C₂₀)alkynyl, (C₁-C₂₀)acyl, (C₁-C₂₀)cycloalkyl, (C₁-C₂₀)aryl, (C₁-C₂₀)alkoxy, (C₁-C₂₀)haloalkyl, and combinations thereof.

13. The method of claim 11, wherein the compound is represented by Formula II: 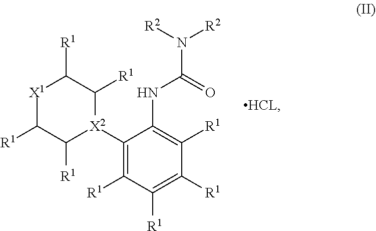
wherein
X¹ is —C—, —N—, or —O—;
X² is —N— or —C—; and
R¹ and R² are independently selected from the group consisting of —H, —F, —OH, substituted or unsubstituted (C₁-C₂₀) hydrocarbyl, and combinations thereof.

14. The method of claim 13, wherein the (C₁-C₂₀)hydrocarbyl is selected from the group consisting of (C₁-C₂₀)alkyl, (C₁-C₂₀)alkenyl, (C₁-C₂₀)alkynyl, (C₁-C₂₀)acyl, (C₁-C₂₀)cycloalkyl, (C₁-C₂₀)aryl, (C₁-C₂₀)alkoxy, (C₁-C₂₀)haloalkyl, and combinations thereof.

15. The method of claim 11, wherein the compound is represented by Formula III: 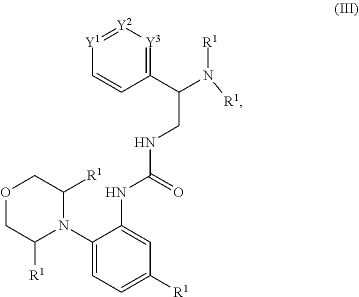
wherein
at each occurrence, R¹ is independently selected from the group consisting of —H, —F, —CF₃, —CH₃, —CH₂CH₃, —OCH₃, —OCH₂CH₃, and combinations thereof; and
Y¹, Y², and Y³ are independently selected from the group consisting of —C—, —N—, —O—, and combinations thereof.

16. The method of claim 11, wherein the compound is represented by Formula IV:
wherein the compound is represented by Formula IV: 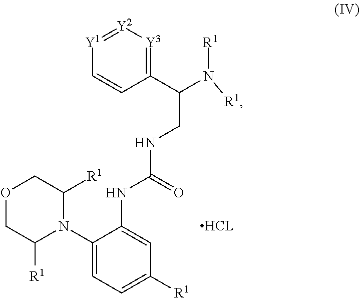
wherein
at each occurrence, R¹ is independently selected from the group consisting of —H, —F, —CF₃, —CH₃, —CH₂CH₃, —OCH₃, —OCH₂CH₃, and combinations thereof; and
Y¹, Y², and Y³ are independently selected from the group consisting of —N—, —C—, —O—, and combinations thereof.

17. The method of claim 11, wherein the compound is represented by Formula V:

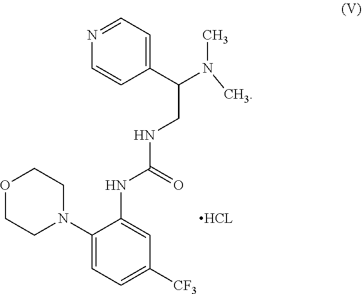

18. The method of claim 11, wherein the compound is represented by Formula VI:
wherein n is any positive integer.

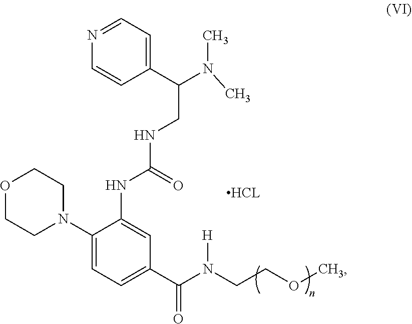

19. The method of claim 11, wherein the compound is represented by Formula VII: 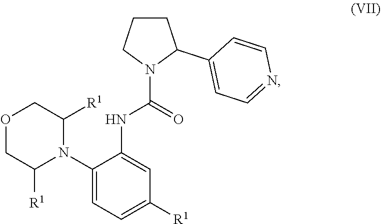
wherein at each occurrence, R¹ is independently selected from the group consisting of —H, —F, —CF₃, —CH₃, —CH₂CH₃, —OCH₃, —OCH₂CH₃, and combinations thereof.

20. The method of claim 11, wherein the compound is represented by Formula VIII: 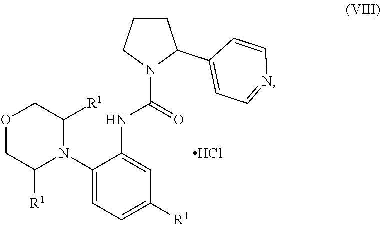
wherein at each occurrence, R¹ is independently selected from the group consisting of —H, —F, —CF₃, —CH₃, —CH₂CH₃, —OCH₃, —OCH₂CH₃, and combinations thereof.
